(19) United States
(12) Patent Application Publication
(10) Pub. No.: US 2021/0148914 A1
(43) Pub. Date: May 20, 2021

(54) TEST FOR DETECTING MALIGNANT KIDNEY CANCER

(71) Applicant: Universität zu Köln, Köln (DE)

(72) Inventors: Melanie von Brandenstein, Lindlar (DE); Jochen Fries, Pulheim (DE)

(73) Assignee: Universität zu Köln, Köln (DE)

(21) Appl. No.: 16/630,416

(22) PCT Filed: Jul. 10, 2018
(86) PCT No.: PCT/EP2018/068713

§ 371 Date: Jan. 10, 2020

(30) Foreign Application Priority Data
Jul. 10, 2017 (EP) 17180479.2

(51) Int. Cl. G01N 33/574; C12Q 1/6886; C07K 16/40; G01N 33/543

(52) U.S. Cl. CPC G01N 33/57438; C12Q 1/6886; C07K 16/40; G01N 2333/912; C12Q 2600/158; C07K 2317/92; G01N 33/54386

(57) ABSTRACT

The present invention relates to a method for detecting malignant kidney cancer in an individual, said method conducted in vitro comprising the steps of providing a body fluid sample, in particular a urine sample, obtained from the individual, and determining one or more biomarker levels selected from the group consisting of the Mxi-2 level, the Vim3 level, the MAPKp38 level and the Atg7 level in the sample. Further, the present invention refers to a kit and a dipstick for use in such method.

The present invention relates to a method for detecting malignant kidney cancer in an individual, said method conducted in vitro comprising the steps of providing a body fluid sample, in particular a urine sample, obtained from the individual, and determining one or more biomarker levels selected from the group consisting of the Mxi-2 level, the Vim3 level, the MAPK p38 level and the Atg7 level in the sample. Further, the present invention refers to a kit and a dipstick for use in such method.

Malignant kidney cancer is a life-threatening disease. More than 200,000 new cases of kidney cancer are diagnosed in the world each year, accounting for just under 2% of all cancers. In order to decide on further treatment strategy including surgical interventions, there is a need for sufficient means for detecting malignant kidney cancer and to distinguish it from benign neoplasia in the kidney.

An oncocytoma is an epithelial neoplasm composed of oncocytes, large eosinophilic cells having small, round, benign-appearing nuclei sometimes associated with large nucleoli and/or with excessive amounts of mitochondria. The treatment of benign oncocytoma differs from that of malignant kidney cancer such as malignant renal carcinoma. For a malignant kidney cancer, initial treatment is most commonly a radical or partial nephrectomy and remains the mainstay of curative treatment. Where the tumor is confined to the renal parenchyma, the 5-year survival rate is 60-70%, but this is lowered considerably where metastases have occurred. It is relatively resistant to radiation therapy and chemotherapy, although some cases respond to immunotherapy. Oncocytomas are benign lesions and metastases are extremely uncommon. As they cannot be confidently distinguished from malignant kidney cancer in some cases pre-operatively, they are surgically resected. If the diagnosis is suspected pre-operatively then surgery by tumor enucleation can be performed. Accordingly, it is important to differentiate benign oncocytoma from malignant kidney cancer, such as malignant renal carcinoma.

Today, it is still a great challenge to detect malignant kidney cancer, in particular renal cell carcinoma (RCC), in a patient. On the one hand, kidney cancer occurs in the inside of an individual's body. On the other hand, even if neoplastic tissue is identified, it is still hard to distinguish between malignant neoplasia and benign oncocytoma.

For a diagnosis by classical means, samples of the identified neoplastic kidney tissue are obtained by surgical means. It is evident that such method bears considerable health risk and stress for the individual. Therefore, methods for detecting malignant neoplasia without the need of surgical means have been considered.

It has been found that detecting certain isoforms of protein kinase C (PKC) such as PKC-alpha and a micro RNA (miR-15a) can be used to partly differentiate between malignant kidney neoplasia and benign oncocytoma (von Brandenstein et al., The American Journal of Pathology, 2012, 180:1787-1797). Furthermore, it has been found that there is also some linkage between malignant kidney neoplasia and changes in cellular signal transduction (cf., Wagner and Nebreda, Nature Reviews Cancer, 2009, 9:537-549; von Brandenstein et al., Life Sciences, 2012, 91:562-571; Chen et al., Scientific Reports, 2015, 5:13470). None of these documents, however, indicate which factor should be detected in practice. U.S. Pat. No. 6,017,692 describes that the mitogen-activated protein kinase 14 (MAPK14) isoform 3 (Mxi-2) can be used as a marker for detecting a malignant cell in a biological sample by means of measuring Mxi gene expression alterations. Such method is, however, based on detecting mRNA levels in cellular samples. This also presupposes the extraction of tissue by surgical means. WO 2014/154686 and EP-A 2784510, respectively, describe a method that is based on the detection of Vimentin variant 3 (Vim3) as a marker for benign oncocytoma. WO 1994/017101 teaches that expression of Mxi may be altered in malignant tissue.

There is, however, so far no suitable marker detectable in an extracorporal body fluid that indicates the presence of malignant kidney cancer, in particular renal cell carcinoma (RCC), or a benign oncocytoma. Accordingly, there is still an unmet need for such marker that provides a positive signal when malignant kidney cancer or a benign oncocytoma occurs in an individual.

Surprisingly, it has been found that the level of Mxi-2 can be determined in a body fluid sample, in particular a urine sample, and can reliably indicate the presence of malignant kidney cancer, in particular RCC, in the individual the sample originates from. The optional combination of the detection of Mxi-2 with other markers such as, e.g., Vim3 indicating the presence of benign oncocytoma even allows differential diagnosis.

Accordingly, the present invention relates to a method for detecting malignant kidney cancer in an individual, said method conducted in vitro comprising the following steps:

(i) providing a body fluid sample obtained from the individual; and

(ii) determining Mxi-2 and/or Vim3 level(s) in said sample.

In general, a body fluid may be any fluid that is provided by an individual's body known in the art that may potentially comprise Mxi-2 and/or Vim3 (or Mxi-2, Vim3, Atg7 and/or MAPK p38). A body fluid may be a fluid excreted from the individual's body or may be a fluid present in the individual's body. When it is a fluid present in the individual's body, it is preferably an extracellular body fluid. Preferably, the body fluid is such that can physiologically get in contact with the kidney. Exemplarily, such body fluid may be urine or a fraction thereof, an intravascular body fluid or a fraction thereof (e.g., blood, blood plasma, blood serum, lymphatic fluid), interstitial body fluid or a fraction thereof, transcellular body fluid or a fraction thereof, or a mixture of two or more thereof. Preferably, the body fluid is urine. Then, the body fluid sample is a urine sample U.

Accordingly, the present invention relates to a method for detecting malignant kidney cancer and/or a (benign kidney) oncocytoma in an individual, said method conducted in vitro, comprising determining one or more biomarker levels selected from the group consisting of the Mxi-2 level, the Vim3 level, the MAPK p38 level and the Atg7 level in a urine sample U from the individual.

Preferably, the present invention relates to a method for detecting malignant kidney cancer and/or a (benign kidney) oncocytoma in an individual, said method conducted in vitro, comprising determining the Mxi-2 level and/or the Vim3 level in a urine sample U from the individual.

The present invention relates to a method for detecting malignant kidney cancer in an individual, said method conducted in vitro comprising the following steps:

(i) providing a urine sample U obtained from the individual; and

(ii) determining Mxi-2 and/or Vim3 level(s) in the urine sample U.

An aspect of the present invention relates to a method for detecting malignant kidney cancer in an individual, said method conducted in vitro, comprising determining the Mxi-2 level in a urine sample U from the individual.

An aspect of the present invention relates to a method for detecting malignant kidney cancer in an individual, said method conducted in vitro comprising the following steps:

(i) providing a urine sample U obtained from the individual; and

(ii) determining Mxi-2 level in the urine sample U.

It will be understood that all specifications, aspects and embodiments described in the context of the urine sample U may apply mutatis mutandis to any body fluid sample in general. This applies particularly to those body fluids that may potentially comprise Mxi-2 and/or Vim3 and/or Atg7 and/or MAPK p38 (in particular Mxi-2 and/or Vim3), in particularly such body fluid that may physiologically get in contact with the kidney. Thus, also a method for detecting malignant kidney cancer in an individual is disclosed, wherein said method is conducted in vitro comprising the following steps:

(i) providing a body fluid sample obtained from the individual; and

(ii) determining Mxi-2 level in the sample.

The method of the present invention may be for determining whether the individual bears malignant kidney cancer, in particular when said individual is suspected for bearing malignant kidney cancer. Exemplarily, an individual is suspected for bearing malignant kidney cancer when a neoplasm has been found in the individual's kidney or is suspected to be present in the individual's kidney.

The person skilled in the art will notice that the method of the present invention is a method not directly associated with the diagnosis of the human or animal body. The result obtained by the method may be used for medical or non-medical purposes. The urine sample U typically is an in vitro specimen, i.e., a specimen remote from the human and animal body.

As used herein, the term “Mxi-2 level” may be understood in the broadest sense, including the Mxi-2 polypeptide level and/or the Mxi-2 mRNA level (typically correlating to the Mxi-2 polypeptide level) in a sample (e.g., the urine sample U or a control sample). The Mxi-2 level may also be understood as Mxi-2 expression level.

As used herein, the term “level” may be understood in the broadest sense as an content of a respective polypeptide or messenger RNA (mRNA) in a sample (e.g., the urine sample U or a control sample). Typically, but not necessarily, such level is related to a reference value such as the sample volume, the total polypeptide or nucleotide content comprised in same sample (e.g., the urine sample U or a control sample) or the content of an intrinsic marker (e.g., transferrin and/or beta-actin), in particular beta-actin, of known concentration naturally contained in the same sample (e.g., the urine sample U or a control sample). If related to the sample volume, the level indicates the concentration, i.e., the respective polypeptide or mRNA per volume (e.g, mass of Mxi-2 polypeptide per volume of the urine sample U [ng/ml]). If related to the total polypeptide content, a relative ratio may be provided (e.g, mass of Mxi-2 polypeptide per mass of total polypeptide content of the urine sample U [pg/ng]). If related to the total nucleotide, a relative ratio may be provided (e.g, mass of Mxi-2 mRNA per mass of total nucleotide content of the urine sample U [pg/ng]). If related to the content of an intrinsic marker, a relative ratio may be provided (e.g, mass of Mxi-2 polypeptide per mass of transferrin [pg/pg]).

Preferably, Mxi-2 is the naturally occurring Mxi-2 of the species of the individual of interest, i.e., the Mxi-2 occurring in the respective urine sample U. Exemplarily, Mxi-2 may be human Mxi-2 or Mxi-2 of a non-human animal, in particular a domestic mammal such as, e.g., a bovine, a pig, a horse, a donkey, a sheep, a goat, a dog, a cat, etc.). In a particularly preferred embodiment, Mxi-2 is human Mxi-2.

Preferably, in the context of the present invention, Mxi-2 has a homology of at least 80%, more preferably at least 90%, even more preferably at least 95%, even more preferably at least 98%, even more preferably at least 99% of SEQ ID NO: 3, in particular is identical with SEQ ID NO: 3:

| | |
|---|---|
| 1 | msqerptfyr qelnktiwev peryqnlspv gsgaygsvca afdtktglrv avkklsrpfq |
| 61 | siihakrtyr elrllkhmkh enviglldvf tparsleefn dvylvthlmg adlnnivkcq |
| 121 | kltddhvqfl iyqilrglky ihsadiihrd lkpsnlavne dcelkildfg larhtddemt |
| 181 | gyvatrwyra peimlnwmhy nqtvdiwsvg cimaelltgr tlfpgtdhid qlklilrlvg |
| 241 | tpgaellkki ssesarnyiq sltqmpkmnf anvfiganpl gkltiyphlm dielvmi |

This sequence reflects the protein sequence NP_620582.1. Human Mxi-2 may also be such as described in U.S. Pat. No. 6,017,692. In an alternative preferred embodiment, Mxi-2 is mammalian non-human Mxi-2 such as, e.g., bovine Mxi-2, pig Mxi-2, horse Mxi-2, donkey Mxi-2, sheep Mxi-2, goat Mxi-2, dog Mxi-2, or cat Mxi-2.

Alternatively, Mxi-2 may be the gene expression product of Homo sapiens mitogen-activated protein kinase 14 (MAPK14), transcript variant 3 (SEQ ID NO: 10):

TTCTCTCACGAAGCCCCGCCCGCGGAGAGGTTCCATATTGGGTAAAATC
TCGGCTCTCGGAGAGTCCCGGGAGCTGTTCTCGCGAGAGTACTGCGGGA
GGCTCCCGTTTGCTGGCTCTTGGAACCGCGACCACTGGAGCCTTAGCGG
GCGCAGCAGCTGGAACGGGAGTACTGCGACGCAGCCCGGAGTCGGCCTT
GTAGGGGCGAAGGTGCAGGGAGATCGCGGCGGGCGCAGTCTTGAGCGCC
GGAGCGCGTCCCTGCCCTTAGCGGGGCTTGCCCCAGTCGCAGGGGCACA
TCCAGCCGCTGCGGCTGACAGCAGCCGCGCGCGCGGGAGTCTGCGGGGT
CGCGGCAGCCGCACCTGCGCGGGCGACCAGCGCAAGGTCCCCGCCCGGC
TGGGCGGGCAGCAAGGGCCGGGGAGAGGGTGCGGGTGCAGGCGGGGGCC
CCACAGGGCCACCTTCTTGCCCGGCGGCTGCCGCTGGAAAATGTCTCAG
GAGAGGCCCACGTTCTACCGGCAGGAGCTGAACAAGACAATCTGGGAGG
TGCCCGAGCGTTACCAGAACCTGTCTCCAGTGGGCTCTGGCGCCTATGG
CTCTGTGTGTGCTGCTTTTGACACAAAAACGGGGTTACGTGTGGCAGTG
AAGAAGCTCTCCAGACCATTTCAGTCCATCATTCATGCGAAAAGAACCT
ACAGAGAACTGCGGTTACTTAAACATATGAAACATGAAAATGTGATTGG
TCTGTTGGACGTTTTTACACCTGCAAGGTCTCTGGAGGAATTCAATGAT
GTGTATCTGGTGACCCATCTCATGGGGGCAGATCTGAACAACATTGTGA
AATGTCAGAAGCTTACAGATGACCATGTTCAGTTCCTTATCTACCAAAT
TCTCCGAGGTCTAAAGTATATACATTCAGCTGACATAATTCACAGGGAC
CTAAAACCTAGTAATCTAGCTGTGAATGAAGACTGTGAGCTGAAGATTC
TGGATTTTGGACTGGCTCGGCACACAGATGATGAAATGACAGGCTACGT
GGCCACTAGGTGGTACAGGGCTCCTGAGATCATGCTGAACTGGATGCAT
TACAACCAGACAGTTGATATTTGGTCAGTGGGATGCATAATGGCCGAGC
TGTTGACTGGAAGAACATTGTTTCCTGGTACAGACCATATTGATCAGTT
GAAGCTCATTTTAAGACTCGTTGGAACCCCAGGGGCTGAGCTTTTGAAG
AAAATCTCCTCAGAGTCTGCAAGAAACTATATTCAGTCTTTGACTCAGA
TGCCGAAGATGAACTTTGCGAATGTATTTATTGGTGCCAATCCCCTGGG
TAAGTTGACCATATATCCTCACCTCATGGATATTGAATTGGTTATGATA
TAAATTGGGGATTTGAAGAAGAGTTTCTCCTTTTGACCAAATAAAGTAC
CATTAGTTGA

Vim3 (also designated as Vimentin3, Vimentin variant 3, Vimentin splice form 3) is a splice isoform of Vimentin. Vim3 in the context of the present invention may be any Vim3 compound. Vimentin itself is an intermediate sized filament that functions in signal transduction cellular function, structural integrity of cells and tissues and adhesion and migration. In 2007, a variant of Vimentin (Vim3) was described by a working group of the Craig Venter Institute (NHLBI Resequencing and Genotyping Service (RSG), N01-NV-48196, J. Craig Venter Institute, Rockville, Md. 20850). In 2011, the presence of this Vim3 in gliomas was described (Thakkar et al., 2011, Cancer Invest 29:113-122). Vim3 is a truncated/spliced variant of Vimentin with a unique C-terminal ending. Preferably, Vim3 is the naturally occurring Vim3 of the species of the individual of interest, i.e., the Vim3 occurring in the respective urine sample U. Exemplarily, Vim3 may be human Vim3 or Vim3 of a non-human animal, in particular a domestic mammal such as, e.g., a bovine, a pig, a horse, a donkey, a sheep, a goat, a dog, a cat, etc.). Vim3 has been found in numerous species so far. In a particularly preferred embodiment, Vim3 is human Vim3.

The human splice variant Vim3 is has 431 amino acids and is 35 amino acids smaller than the full length protein. Its unique structure leads to a 10 kDa smaller protein. The amino acid sequence of human Vim3 has been published and is available at UniProt KB (http://www.uniprot.org/uniprot/BOYJC4) or at the National Center for Biotechnology Information under GenBank Accession number ACA06103.1 (http://www.ncbi.nlm.nih.gov/protein/167887751). Preferably, in the context of the present invention, Vim3 has a homology of at least 80%, more preferably at least 90%, even more preferably at least 95%, even more preferably at least 98%, even more preferably at least 99% of SEQ ID NO: 1, in particular is identical with SEQ ID NO: 1:

| | |
|---|---|
| 1 | mstrsvssss yrrmfggpgt asrpsssrsy vttstrtysl gsalrpstsr slyasspggv |
| 61 | yatrssavrl rssvpgvrll qdsvdfslad aintefkntr tnekvelqel ndrfanyidk |
| 121 | vrfleqqnki llaeleqlkg qgksrlgdly eeemrelrrq vdqltndkar veverdnlae |
| 181 | dimrlreklq eemlqreeae ntlqsfrqdv dnaslarldl erkveslqee iaflkklhee |
| 241 | eiqelqaqiq eqhvgidvdv skpdltaalr dvrqqyesva aknlqeaeew ykskfadlse |
| 301 | aanrnndalr qakqesteyr rqvqsltcev dalkgtnesl erqmremeen faveaanyqd |
| 361 | tigrlqdeiq nmkeemarhl reyqdllnvk maldieiaty rkllegeesr islplpnfss |
| 421 | lnlrgkhfis l |

In an alternative preferred embodiment, Vim3 is mammalian non-human Vim3 such as, e.g., bovine Vim3, pig Vim3, horse Vim3, donkey Vim3, sheep Vim3, goat Vim3, dog Vim3, or cat Vim3. The Vimentin sequence of other species is also known, including e.g. Mus musculus (NCBI Accession: CAA39807.1, NP_035831.2), Rattus norvegicus (NCBI Accession: NP_112402.1), Bos taurus (NCBI Accession: NP_776394.2), Gallus gallus (NCBI Accession: NP_001041541.1), Mesocricetus auratus (Accession: AAA37104.1), Oncorhynchus mykiss (Accession: CAA90601.1), Equus caballus (NP_001230074.1), Salmo salar (Accession: NP_001133947.1), Pan troglodytes (Accession: NP_001009148.1) and Cavia porcellus (Accession: NP_001166511.1). The splice variant corresponding to human Vim3 could be easily identified by sequence analysis and identification of homologues.

Preferably, in the context of the present invention, Atg7 (Ubiquitin-like modifier-activating enzyme ATG7) has a homology of at least 80%, more preferably at least 90%, even more preferably at least 95%, even more preferably at least 98%, even more preferably at least 99% or sequence identity of the polypeptide of the UniProtKB database No. 095352 (ATG7_HUMAN). Preferably, in the context of the present invention, Atg7 has a homology of at least 80%, more preferably at least 90%, even more preferably at least 95%, even more preferably at least 98%, even more preferably at least 99% or sequence identity of SEQ ID NO: 11:

MAAATGDPGLSKLQFAPFSSALDVGFWHELTQKKLNEYRLDEAPKDIKG
YYYNGDSAGLPARLTLEFSAFDMSAPTPARCCPAIGTLYNTNTLESFKT
ADKKLLLEQAANEIWESIKSGTALENPVLLNKFLLLTFADLKKYHFYYW
FCYPALCLPESLPLIQGPVGLDQRFSLKQIEALECAYDNLCQTEGVTAL
PYFLIKYDENMVLVSLLKHYSDFFQGQRTKITIGVYDPCNLAQYPGWPL
RNFLVLAAHRWSSSFQSVEVVCFRDRTMQGARDVAHSIIFEVKLPEMAF
SPDCPKAVGWEKNQKGGMGPRMVNLSECMDPKRLAESSVDLNLKLMCWR
LVPTLDLDKVVSVKCLLLGAGTLGCNVARTLMGWGVRHITFVDNAKISY
SNPVRQPLYEFEDCLGGGKPKALAAADRLQKIFPGVNARGFNMSIPMPG
HPVNFSSVTLEQARRDVEQLEQLIESHDVVFLLMDTRESRWLPAVIAAS
KRKLVINAALGFDTFVVMRHGLKKPKQQGAGDLCPNHPVASADLLGSSL
FANIPGYKLGCYFCNDVVAPGDSTRDRTLDQQCTVSRPGLAVIAGALAV
ELMVSVLQHPEGGYAIASSSDDRMNEPPTSLGLVPHQIRGFLSRFDNVL
PVSLAFDKCTACSSKVLDQYEREGFNFLAKVFNSSHSFLEDLTGLTLLH
QETQAAEIWDMSDDETI

In an alternative preferred embodiment, Atg7 is a truncated version of Atg7, e.g., truncated by one, two or more, up to ten, up to 20, up to 50 or up to 100 amino acid moieties or more than 100 amino acid moieties. In an alternative preferred embodiment, Atg7 is mammalian non-human Atg7 such as, e.g., bovine Atg7, pig Atg7, horse Atg7, donkey Atg7, sheep Atg7, goat Atg7, dog Atg7, or cat Atg7.

Preferably, in the context of the present invention, MAPK p38 (Mitogen-activated protein kinase 14) has a homology of at least 80%, more preferably at least 90%, even more preferably at least 95%, even more preferably at least 98%, even more preferably at least 99% or sequence identity of a polypeptide sequence of the UniProtKB database No. Q16539 (MK14_HUMAN). Preferably, in the context of the present invention, MAPK p38 has a homology of at least 80%, more preferably at least 90%, even more preferably at least 95%, even more preferably at least 98%, even more preferably at least 99% or sequence identity of SEQ ID NO: 12:

MSQERPTFYRQELNKTIWEVPERYQNLSPVGSGAYGSVCAAFDTKTGLR
VAVKKLSRPFQSIIHAKRTYRELRLLKHMKHENVIGLLDVFTPARSLEE
FNDVYLVTHLMGADLNNIVKCQKLTDDHVQFLIYQILRGLKYIHSADII
HRDLKPSNLAVNEDCELKILDFGLARHTDDEMTGYVATRWYRAPEIMLN
WMHYNQTVDIWSVGCIMAELLTGRTLFPGTDHIDQLKLILRLVGTPGAE
LLKKISSESARNYIQSLTQMPKMNFANVFIGANPLAVDLLEKMLVLDSD
KRITAAQALAHAYFAQYHDPDDEPVADPYDQSFESRDLLIDEWKSLTYD
EVISFVPPPLDQEEMES

In an alternative preferred embodiment, MAPK p38 is a truncated version of MAPK p38, e.g., truncated by one, two or more, up to ten, up to 20, up to 50 or up to 100 amino acid moieties or more than 100 amino acid moieties. In an alternative preferred embodiment, MAPK p38 is mammalian non-human MAPK p38 such as, e.g., bovine MAPK p38, pig MAPK p38, horse MAPK p38, donkey MAPK p38, sheep MAPK p38, goat MAPK p38, dog MAPK p38, or cat MAPK p38.

Malignant kidney cancer may be any cancer of the kidney that bears malignancy. Exemplarily, malignant kidney cancer may be selected from the group consisting of renal cell carcinoma (RCC), transitional cell carcinoma (TCC, urothelial cell carcinoma, renal pelvis carcinoma), squamous cell carcinoma, juxtaglomerular cell tumor (reninoma), angiomyolipoma, Bellini duct carcinoma, clear-cell sarcoma of the kidney, mesoblastic nephroma, Wilms' tumor, mixed epithelial stromal tumor, clear cell adenocarcinoma, transitional cell carcinoma, inverted papilloma, renal lymphoma, teratoma, carcinosarcoma and carcinoid tumor of the renal pelvis. Preferably, the malignant kidney cancer is a carcinoma type of renal cancer.

In a preferred embodiment, the malignant kidney cancer is renal cell carcinoma (RCC).

Renal cell carcinoma (RCC) is one type of malignant kidney cancer. Thus, the present invention relates to a method for detecting renal cell carcinoma (RCC) in an individual, said method conducted in vitro comprising the following steps:

(i) providing a urine sample U obtained from the individual; and

(ii) determining Mxi-2 level in the urine sample U.

RCC may exemplarily be selected from the group consisting of clear cell RCC (e.g., clear cell papillary RCC, clear cell RCC with smooth muscle stroma, multilocular cystic clear cell RCC), papillary RCC, chromophobe RCC (including hybrid oncocytoma/chromophobe RCC), mucinous tubular and spindle cell carcinoma, tubulocystic RCC, thyroid-like follicular RCC, acquired cystic kidney disease-associated RCC, RCC with t(6;11) translocation, and hereditary leiomyomatosis and RCC. Preferably, RCC is selected from the group consisting of clear cell RCC, papillary RCC, and chromophobe RCC.

In a preferred embodiment, the malignant kidney cancer is renal cell carcinoma (RCC) bearing a regression rate of <70% or <60%.

The urine sample U may be obtained by any means. Preferably, it is obtained by the act of urination of an individual of interest. The method of the present invention may be conducted during the act of urination (e.g., by contacting a dipstick with the urine stream or urine freshly introduced into a vessel/hollow ware) or may be conducted using a stored urine sample. Storage may be storage of up to 15 min, up to 30 min, up to one hour, up to twelve hours, up to a day, up to a week, up to a month, up to a year or even longer. Long-term storage for more than one day is preferably conducted under any conditions maintaining detectability of the level of the polypeptides of interest (e.g., Mxi-2 and optionally Vim3) such as, e.g., by means of freezing, shock-freezing (e.g., in liquid nitrogen), freeze-draying, and/or the addition of one or more preservative agents, in particular biocide/antimicrobial agents, to the urine sample U.

In the context of the present invention, the terms “polypeptide” and “protein” may be understood interchangeably in the broadest sense as a compound mainly composed of natural amino acid moieties consecutively conjugated with another via amide bonds. It will be understood that a protein in the sense of the present invention may or may not be subjected to one or more posttranslational modification(s) and/or be conjugated with one or more non-amino acid moiety/moieties. The termini of the protein may, optionally, be capped by any means known in the art, such as, e.g., amidation, acetylation, methylation, acylation. Posttranslational modifications are well-known in the art and may be but may not be limited to lipidation, phosphorylation, sulfatation, glycosylation, truncation, oxidation, reduction, decarboxylation, acetylation, amidation, deamidation, disulfide bond formation, amino acid addition, cofactor addition (e.g., biotinylation, heme addition, eicosanoid addition, steroid addition) and complexation of metal ions, non-metal ions, peptides or small molecules and addition of iron-sulphide clusters. Moreover, optionally, co-factors, in particular cyclic guanidinium monophosphate (cGMP), but optionally also such as, e.g., ATP, ADP, NAD⁺, NADH+H⁺, NADP⁺, NADPH+H⁺, metal ions, anions, lipids, etc. may be bound to the protein, irrespective on the biological influence of these co-factors.

It will be understood that such polypeptide may also bear one or more non-natural amino acid moiety/moieties and/or one or more posttranscriptional modification(s) and/or may be conjugated to one or more further structures such as label moieties (e.g., by means of a dye (e.g., a fluorescence dye) or a metal label (e.g., gold beads)). In the context of Mxi-2 and if detected Vim3, the respective polypeptide is preferably the one generated by the individual of interest.

An individual of interest from which the urine sample U is obtained from may be a human or non-human animal. A non-human animal preferably is a non-human mammal, in particular a domestic mammals such as, e.g., a bovine, a pig, a horse, a donkey, a sheep, a camel, a goat, a dog, a cat, etc. Preferably, the individual of interest from which the urine sample U is obtained from is a human. The individual, in particular when it is a human, may also be designated as patient.

In a preferred embodiment, the individual is known to comprise at least one neoplasm of kidney tissue.

In one preferred embodiment, but not necessarily, the individual is known to comprise at least one neoplasm originating from proximal convoluted tubule of the kidney.

In this context, the individual preferably is known or at least suspected to comprise at least one neoplasm of kidney tissue which is unknown to be benign or malign. More preferably, the individual is known to comprise at least one neoplasm of kidney tissue which is assumed to be either malignant renal cell carcinoma (RCC) or benign oncocytoma.

Preferably, the level of the polypeptide(s) of interest such as Mxi-2 (and optionally additionally Vim3, Atg7 and/or MAPK p38, in particular additionally Vim3) is conducted by determining the level directly in the urine sample U. Alternatively, an aliquot of the urine sample U may be diluted in a liquid that maintains the detectability of the levels of the polypeptides of interest such as Mxi-2 and optionally Vim3, Atg7 and/or MAPK p38, in particular additionally Vim3 (e.g., by means of an aqueous buffer and/or an organic solvent (e.g., dimethyl sulfoxide)).

The step of determining Mxi-2 levels in the urine sample U may be conducted by any means. Preferably, the step of determining the Mxi-2 level is determining the level of Mxi-2 polypeptide and/or the level of Mxi-2 messenger RNA (mRNA), in particular the Mxi-2 polypeptide. Determining the level of a polypeptide typically bears the advantage that polypeptides are rather stable in body fluids such as urine. Therefore, the level of Mxi-2 polypeptide can be determined in the urine sample U without any burden.

In a preferred embodiment, the step of determining the Mxi-2 level is determining the level of Mxi-2 polypeptide.

In order to improve comparability of different urine samples U with another, in other words to normalize the determined results, the level of Mxi-2 polypeptide determined in a sample is preferably related to a reference value such as the sample volume, the total polypeptide content comprised in same sample (e.g., the urine sample U or a control sample) or the content of an intrinsic marker (e.g., transferrin and/or beta-actin) of known concentration naturally contained in the same sample (e.g., the urine sample U or a control sample). If related to the sample volume, the level indicates the concentration, i.e., the respective polypeptide per volume (e.g, mass of Mxi-2 polypeptide per volume of the urine sample U [ng/ml]). If related to the total polypeptide content, a relative ratio may be provided (e.g, mass of Mxi-2 polypeptide per mass of total polypeptide content of the urine sample U [pg/ng]). If related to the content of an intrinsic marker, a relative ratio may be provided (e.g, mass of Mxi-2 polypeptide per mass of transferrin [pg/pg] or the mass of Mxi-2 polypeptide per mass of beta-actin [pg/pg]).

Highly preferably, the step of determining the Mxi-2 level is determining the level of Mxi-2 polypeptide in relation to the total polypeptide content comprised in the respective sample.

Highly preferably, the present invention relates to a method for detecting renal cell carcinoma (RCC) in an individual, said method conducted in vitro comprising the following steps:

(i) providing a urine sample U obtained from the individual; and

(ii) determining the level of Mxi-2 polypeptide in the urine sample U.

In an alternative preferred embodiment, the step of determining the Mxi-2 level is determining the level of Mxi-2 messenger RNA (mRNA).

In order to improve comparability of different urine samples U with another, in other words to normalize the determined results, the level of Mxi-2 mRNA determined in a sample is preferably related to a reference value such as the sample volume, the total nucleotide or mRNA content comprised in same sample (e.g., the urine sample U or a control sample) or the amount of an intrinsic marker (e.g., transferrin and/or beta-actin polypeptide or transferrin and/or beta-actin mRNA) of known concentration naturally contained in the same sample (e.g., the urine sample U or a control sample). If related to the sample volume, the level indicates the concentration, i.e., the respective mRNA per volume (e.g, mass of Mxi-2 mRNA per volume of the urine sample U [ng/ml]). If related to the total nucleotide or mRNA content, a relative ratio may be provided (e.g, mass of Mxi-2 mRNA per mass of total nucleotide or mRNA content of the urine sample U [pg/ng]). If related to the content of an intrinsic marker, a relative ratio may be provided (e.g, mass of Mxi-2 mRNA per mass of transferrin [pg/pg] or the mass of Mxi-2 mRNA per mass of beta-actin [pg/pg], or Mxi-2 mRNA per mass of transferrin mRNA [pg/pg] or the mass of Mxi-2 mRNA per mass of beta-actin mRNA [pg/pg].

Highly preferably, the step of determining the Mxi-2 level is determining the level of Mxi-2 mRNA in relation to the total polypeptide, the total nucleotide, or the total mRNA content comprised in the respective sample.

As indicated above, one of the advantages of the present invention is that a malignant kidney cancer, preferably a carcinoma type of renal cancer, in particular renal cell carcinoma (RCC), can be very well detected by the method of the present invention.

Accordingly, in a preferred embodiment, an increased Mxi-2 level indicates the presence of malignant kidney cancer in the individual.

Highly preferably, the present invention relates to a method for detecting renal cell carcinoma (RCC) in an individual, said method conducted in vitro comprising the following steps:

(i) providing a urine sample U obtained from the individual; and

(ii) determining the level of Mxi-2 polypeptide in the urine sample U,

Wherein an the level of Mxi-2 polypeptide indicates the presence of RCC in the individual.

In order to improve comparability of different urine samples U with another, in other words to normalize the determined and to improve reproducibility of the measurements, the determined Mxi-2 level determined in step (ii) is compared to a reference value. Such reference value may be an internal control (i.e., a further control sample C measured under comparable conditions, preferably in the same test series) or may be a predetermined reference value R1 typically but not necessarily obtained from one or more previous measurements conducted under comparable conditions.

Accordingly, in a preferred embodiment, the method further comprises the step of

(iii) comparing the Mxi-2 level determined in step (ii) with
(a) a predetermined reference value R1 indicating the borderline between a sample indicating the presence of malignant kidney cancer and a sample indicating the absence of malignant kidney cancer; and/or
(b) Mxi-2 level determined in a control sample C obtained from a control individual of the same species free of malignant cancer cells,

wherein an Mxi-2 level determined in the urine sample U that is higher than R1 and/or at least 20% higher than the Mxi-2 level of C indicates the presence of malignant kidney cancer in the individual,

wherein the Mxi-2 level in each case is related to the total polypeptide content comprised in the respective sample.

Throughout the present application, it will be understood that when the malignant kidney cancer is renal cell carcinoma (RCC), the malignant cancer cells are preferably renal carcinoma cells.

Highly preferably, the present invention relates to a method for detecting renal cell carcinoma (RCC) in an individual, said method conducted in vitro comprising the following steps:

(i) providing a urine sample U obtained from the individual;

(ii) determining the level of Mxi-2 polypeptide in the urine sample U; and

(iii) comparing the level of Mxi-2 polypeptide determined in step (ii) with
(a) a predetermined reference value R1 indicating the borderline between a sample indicating the presence of RCC and a sample indicating the absence of RCC; and/or
(b) level of Mxi-2 polypeptide determined in a control sample C obtained from a control individual of the same species free of renal carcinoma cells,

wherein an level of Mxi-2 polypeptide determined in the urine sample U that is higher than R1 and/or at least 20% higher than the level of Mxi-2 polypeptide of C indicates the presence of RCC in the individual,

wherein the level of Mxi-2 polypeptide in each case is related to the total polypeptide content comprised in the respective sample.

The optional combination of the determination of the level of Mxi-2 with the level of other markers such as, e.g., Vim3 indicating the presence of benign oncocytoma, may improve reliability of the method even further and optionally allows differential diagnosis.

Accordingly, in a preferred embodiment, the method further comprises the step of: (iv) determining of Vim3 level in the urine sample U.

As used herein, the term “Vim3 level” may be understood in the broadest sense, including the Vim3 polypeptide level and/or the Vim3 mRNA level (directly correlating to the Vim3 polypeptide level) in a sample (e.g., the urine sample U or a control sample). The Vim3 level may also be understood as Vim3 expression level.

In a more preferred embodiment, determining the Vim3 level (step iv) is determining the level of Vim3 polypeptide.

In an alternative preferred embodiment, determining the Vim3 level (step iv) is determining the level of Vim3 messenger RNA (mRNA).

In order to improve comparability of different urine samples U with another, in other words to normalize the determined results, the level of Vim3 polypeptide or Vim3 mRNA determined in a sample is preferably related to a reference value. This is to be understood mutatis mutandis as laid out in the context of Mxi-2 above.

Accordingly, if the Vim3 level (step iv) is determining the level of Vim3 polypeptide, this determined level may be related to a reference value such as the sample volume, the total polypeptide content comprised in same sample (e.g., the urine sample U or a control sample) or the content of an intrinsic marker (e.g., transferrin and/or beta-actin) of known concentration naturally contained in the same sample (e.g., the urine sample U or a control sample). If related to the sample volume, the level indicates the concentration, i.e., the respective polypeptide per volume (e.g, mass of Vim3 polypeptide per volume of the urine sample U [ng/ml]). If related to the total polypeptide content, a relative ratio may be provided (e.g, mass of Vim3 polypeptide per mass of total polypeptide content of the urine sample U [pg/ng]). If related to the content of an intrinsic marker, a relative ratio may be provided (e.g, mass of Vim3 polypeptide per mass of transferrin [pg/pg] or the mass of Vim3 polypeptide per mass of beta-actin [pg/pg]).

If the Vim3 level (step iv) is determining the level of Vim3 mRNA, this determined level may be related to a reference value such as the sample volume, the total nucleotide or mRNA content comprised in same sample (e.g., the urine sample U or a control sample) or the amount of an intrinsic marker (e.g., transferrin and/or beta-actin polypeptide or transferrin and/or beta-actin mRNA) of known concentration naturally contained in the same sample (e.g., the urine sample U or a control sample). If related to the sample volume, the level indicates the concentration, i.e., the respective mRNA per volume (e.g, mass of Vim3 mRNA per volume of the urine sample U [ng/ml]). If related to the total nucleotide or mRNA content, a relative ratio may be provided (e.g, mass of Vim3 mRNA per mass of total nucleotide or mRNA content of the urine sample U [pg/ng]). If related to the content of an intrinsic marker, a relative ratio may be provided (e.g, mass of Vim3 mRNA per mass of transferrin [pg/pg] or the mass of Vim3 mRNA per mass of beta-actin [pg/pg], or Vim3 mRNA per mass of transferrin mRNA [pg/pg] or the mass of Vim3 mRNA per mass of beta-actin mRNA [pg/pg].

It will be understood that the specifications made in the context of the methodical steps of detecting Mxi-2 as described above apply mutatis mutandis to the methodical steps of detecting Vim3 instead, except that the presence, in particular an increased level, of Vim3 does not indicate malignancy.

In contrary, in a preferred embodiment, in step (iv), a decreased Vim3 level, in particular the absence of Vim3, indicates the presence of malignant kidney cancer in the individual.

In a more preferred embodiment, the method further comprises the step of:

(v) comparing the Vim3 level determined in step (iv) with
(a) a predetermined reference value R2 indicating the borderline between a sample indicating the presence of malignant kidney cancer and a sample indicating the absence of malignant kidney cancer; and/or
(b) Vim3 level determined in the control sample C,

wherein an Vim3 level determined in the urine sample U that is lower than R2 and/or at least 50% lower than the Vim3 level of C, in particular the absence of Vim3, indicates the presence of malignant kidney cancer in the individual, wherein the Vim3 level in each case is related to the total polypeptide content comprised in the respective sample.

Highly preferably, the present invention relates to a method for detecting renal cell carcinoma (RCC) in an individual, said method conducted in vitro comprising the following steps:
(i) providing a urine sample U obtained from the individual;
(ii) determining the level of Mxi-2 polypeptide in the urine sample U; and
(iii) optionally comparing the level of Mxi-2 polypeptide determined in step (ii) with
   (a) a predetermined reference value R1 indicating the borderline between a sample indicating the presence of RCC and a sample indicating the absence of RCC; and/or
   (b) level of Mxi-2 polypeptide determined in a control sample C obtained from a control individual of the same species free of renal carcinoma cells; and
(iv) determining the Vim3 level (step iv) is determining the level of Vim3 polypeptide;
(v) optionally comparing the level of Vim3 polypeptide determined in step (iv) with
   (a) a predetermined reference value R2 indicating the borderline between a sample indicating the presence of RCC and a sample indicating the absence of RCC; and/or
   (b) level of Vim3 polypeptide determined in the control sample C,

optionally wherein an level of Mxi-2 polypeptide determined in the urine sample U that is higher than R1 and/or at least 20% higher than the level of Mxi-2 polypeptide of C indicates the presence of RCC in the individual,

wherein the level of Mxi-2 polypeptide in each case is related to the total polypeptide content comprised in the respective sample;

optionally wherein a level of Vim3 polypeptide determined in the urine sample U that is lower than R2 and/or at least 50% lower than the Level of Vim3 polypeptide of C, in particular the absence of Vim3, indicates the presence of malignant kidney cancer in the individual, wherein the level of Vim3 polypeptide in each case is related to the total polypeptide content comprised in the respective sample.

As indicated above, determining the level of Mxi-2 and concomitantly the level of Vim3 bears the advantage that a differential diagnosis is enabled. This can be particularly well performed by determining the level ratio Mxi-2:Vim3.

Therefore, in a preferred embodiment, an increase of the level ratio Mxi-2:Vim3 in comparison to a predetermined reference value R3 indicating the borderline between a sample indicating the presence of malignant kidney cancer and a sample indicating the absence of malignant kidney cancer indicates the presence of malignant kidney cancer in the individual.

The level ratio Mxi-2:Vim3 enables to obtain a particularly beneficial diagnostic information obtainable from the in vitro method of the present invention.

In a more preferred embodiment, the individual is known to comprise either

(a) malignant renal cell carcinoma (RCC), or

(b) benign oncocytoma,

and the method is conducted to differentiate between (a) and (b), wherein (a) is characterized by an increase of the level ratio Mxi-2:Vim3.

As indicated above, step (ii) of the method of the present invention (referring to determining Mxi-2 level in the urine sample U and if present step (iv) of the present invention (referring to determining of Vim3 level in the urine sample U) may be performed by any means.

In a preferred embodiment, the wherein step (ii) and if present step (iv) comprises at least one of:
a) determining respective polypeptide level of Mxi-2 and/or Vim3 by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof; and/or
b) determining the respective messenger RNA (mRNA) level of Mxi-2 and/or Vim3 by means of conducting at least one step selected from the group consisting of polymerase chain reaction (PCR), real time PCR (RT-PCR), by in situ hybridization, gel electrophoresis, Northern Blot, Southern Blot, and combinations of two or more thereof.

In a preferred embodiment, the wherein step (ii) and if present step (iv) comprises at least one of:
a) determining respective polypeptide level of Mxi-2 and/or Vim3 by means of conducting at least one step selected from the group consisting of enzyme-linked immunosorbent assay (ELISA), immuno-electrophoresis, immuno-blotting, Western blot, capillary electrophoresis (CE), spectrophotometry or enzyme assay for example, dipsticks (lateral flow), and combinations of two or more thereof; and/or
b) determining the respective messenger RNA (mRNA) level of Mxi-2 and/or Vim3 by means of conducting at least one step selected from the group consisting of polymerase chain reaction (PCR), real time PCR (RT-PCR), by in situ hybridization, gel electrophoresis, Northern Blot, and combinations of two or more thereof.

Particularly preferably, step (ii) (and if present step (iv)) comprises at least one of determining the respective polypeptide level of Mxi-2 (and if present Vim3) by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof.

An ELISA may be understood in the broadest sense. In a preferred embodiment, ELISA plates on which the polypeptides from the body fluid sample, in particular the urine sample U, can bind may be used. Optionally, the wells may be washed. Then, the body fluid sample, in particular the urine sample U, may be added and incubated to enable binding to the surface. Optionally, the plate may be washed again. Then, a primary antibody or antigen-binding fragment may be added (i.e., e.g. an antibody or antigen-binding fragment binding to Mxi-2, a Vim3, an Atg7 or an MAPK p38) and incubated to enable binding to its target structures. Optionally, the wells may be washed. A labelled secondary antibody or antigen-binding fragment binding to the respective primary antibody or antigen-binding fragment may be added. Optionally, the plate may be washed again. The signal intensity of the label of the secondary antibody or antigen-binding fragment may be determined and quantified. Optionally, the determined value (e.g., absorbance value) may be compared to a reference control (e.g., an empty well, an average value, or a negative or positive sample or a predetermined reference value). An example is provided in the Example section below. It will be understood that, alternatively, also ELISA plates on which primary antibodies or antigen-binding fragments have been coated may be used for an ELISA.

Alternatively, step (ii) (and if present step (iv)) comprises determining the respective mRNA. The latter may be conducted equivalent to the process as described by von Brandenstein et al. in 2012 for PKC-alpha (cf., The American Journal of Pathology, 2012, 180:1787-1797).

Vim3 mRNA may be also detected as described in WO 2014/154686, in particular by means of PCR techniques based on the primers described therein.

In a highly preferred embodiment, the method of the present invention is a method for detecting renal cell carcinoma (RCC) in an individual, said method conducted in vitro comprising the following steps:

(i) providing a urine sample U obtained from the individual;

(ii) determining Mxi-2 level in U;

(iii) comparing the level of Mxi-2 determined in step (ii) with
(a) a predetermined reference value R1 indicating the borderline between a sample indicating the presence of malignant kidney cancer and a sample indicating the absence of malignant kidney cancer, and/or
(b) the level of Mxi-2 in a control sample C obtained from a control individual of the same species free of malignant cancer cells;

(iv) optionally determining the level of Vim3 in U,

(v) optionally comparing the level of Vim3 determined in step (iv) with
(a) a predetermined reference value R2 indicating the borderline between a sample indicating the presence of malignant kidney cancer and a sample indicating the absence of malignant kidney cancer; and/or
(b) the level of Vim3 in C,

wherein the presence of malignant kidney cancer in the individual is indicated by
(I) an level of Mxi-2 determined in U that is higher than R1 and/or at least 20% higher than the level of Mxi-2 in C; and
(II) optionally an level of Vim3 determined in U that is lower than R2 and/or at least 50% lower than the level of Vim3 in C, in particular the absence of Vim3,

preferably wherein the level of Mxi-2 and, if present Vim3, in each case is related to the total polypeptide content comprised in the respective sample.

In addition or alternatively to the Mxi-2 level, also one or more other biomarker levels such as the Vim3 level and/or the Atg7 level and/or the MAPK p38 level can be determined. The (preferred) embodiments described for Mxi-2 herein are mutatis mutandis conductible with Vim3, Atg7, MAPK p38 or a combination of two or more of Mxi-2, Vim3, Atg7 and/or MAPK p38.

Even more preferably, the level of Mxi-2 (and if present Vim3) in the urine sample U may be determined by means of immunodetection.

In a particularly preferred embodiment, the method of the present invention is a method for detecting renal cell carcinoma (RCC) in an individual, said method conducted in vitro comprising the following steps:
(i) providing a urine sample U obtained from the individual;
(ii) determining the level of Mxi-2 polypeptide in U;
(iii) comparing the level of Mxi-2 polypeptide determined in step (ii) with
   (a) a predetermined reference value R1 indicating the borderline between a sample indicating the presence of malignant kidney cancer and a sample indicating the absence of malignant kidney cancer, and/or
   (b) the level of Mxi-2 polypeptide in a control sample C obtained from a control individual of the same species free of malignant cancer cells;
(iv) optionally determining the level of Vim3 polypeptide in U,
(v) optionally comparing the level of Vim3 polypeptide determined in step (iv) with
   (a) a predetermined reference value R2 indicating the borderline between a sample indicating the presence of malignant kidney cancer and a sample indicating the absence of malignant kidney cancer; and/or
   (b) the level of Vim3 polypeptide in C,

wherein the presence of malignant kidney cancer in the individual is indicated by
(I) an level of Mxi-2 polypeptide determined in U that is higher than R1 and/or at least 20% higher than the level of Mxi-2 polypeptide in C; and
(II) optionally an level of Vim3 polypeptide determined in U that is lower than R2 and/or at least 50% lower than the level of Vim3 polypeptide in C, in particular the absence of Vim3,

wherein the level of Mxi-2 polypeptide and, if present Vim3 polypeptide, in each case is related to the total polypeptide content comprised in the respective sample.

Particularly preferably, the level of Mxi-2 polypeptide (and if present Vim3 polypeptide)—optionally alternatively Mxi-2 mRNA (and if present Vim3 mRNA) in the urine sample U may be determined by means of immunodetection. Immunodetection is a particularly reliable and suitable mean for determining levels of polypeptides and is also suitable for detecting nucleotides such as mRNA.

Optionally, the method of the present invention may also be combined with any other means suitable for assessing the presence of malignant kidney cancer, in particular other means suitable for discriminating between malignant kidney cancer and benign neoplasia (in particular between malignant renal cell carcinoma (RCC) and benign oncocytoma). Exemplarily, the method of the present invention may also be combined with the determination of the protein kinase C (PKC) level and/or presence of certain isoforms thereof, such as PKC-alpha, as laid out in WO 2014/154686 and/or by von Brandenstein et al., 2012 (The American Journal of Pathology 180:1787-1797). It will be understood that the specifications made in the context of the method of detecting Mxi-2 and Vim3 as described herein apply mutatis mutandis to the method of detecting PKC and/or certain isoforms thereof.

In a preferred embodiment, the wherein step (ii) and if present step (iv) comprises staining of the respective polypeptide Mxi-2 and/or Vim3, preferably by means of:
(iia) direct immunodetection comprising providing at least one labeled antibody or antibody fragment (in the following designated as AB1-L) specific for the respective polypeptide, and
   enabling the binding of said AB1-L to the respective polypeptide; or
(iib) indirect immunodetection comprising providing at least one unlabeled antibody or antibody fragment (in the following designated as AB1-ul) specific for the respective polypeptide and at least one labeled antibody or antibody fragment (in the following designated as AB2-L) specifically binding to AB1-ul,
   enabling the binding of AB1-ul to the respective polypeptide, and
   enabling the binding of AB2-L to AB1-ul.

It will be understand that the designations AB1-L, AB1-ul and AB2-L are names for the respective antibodies used in order to improve readability. These names, as such, are no technical characterization and can, thus, be omitted in the wording.

As indicated above, alternatively or additionally a corresponding method may also be conducted by determining the level of Vim3 and does not necessarily include concomitantly determining the level of Mxi-2.

Preferred characteristics of a Vim3-specific antibody or fragment or variant thereof are described in WO 2014/154686. Preferably, in the context of Vim3, the antibody or antibody fragment binds to Vim3 with an at least 10-fold, even more preferably at least 100-fold, even more preferably at least 1000-fold higher binding affinity than to full-length Vimentin (V9). In a particularly preferred embodiment, the antibody or antibody fragment binds to the unique C-terminal 8 amino acids of Vim3 (RGKHFISL: SEQ ID No: 2) and/or unique C-terminal 10 amino acids of Vim3 (NLRGKHFISL: SEQ ID NO: 5). This is further exemplified in WO 2014/154686 and in the example section.

In a highly preferred embodiment, an Mxi-2 antibody usable in the context of the method of the present invention specifically binds to the epitope present at a region of Mxi-2 having the sequence GKLTIYPHLMDIELVMI (SEQ ID NO: 4).

In a preferred embodiment, the method of the present invention comprises a further step of treating the individual bearing (and optionally suffering from) malignant kidney cancer.

As used herein, the term “treating” may be understood in the broadest sense. The treating step as described herein may be the sole treatment of the individual or may be combined with one or more other treatment(s) of the individual.

In a preferred embodiment, treating the neoplasm is or comprises administering the individual with an amount of an antineoplastic agent sufficient for treating the type of neoplasm identified in the preceding step.

Accordingly, in a preferred embodiment, the method of the present invention comprises a further step of administering the individual bearing (and optionally suffering from) malignant kidney cancer with an amount of an antineoplastic agent sufficient for treating malignant kidney cancer.

As used herein, the terms “antineoplastic agent”, “anticancer agent”, “antineoplastic drug”, “anticancer drug”, “anticancer compound”, “antineoplastic compound” and equivalents be understood interchangeably in the broadest sense as any agent that is suitable for treating malignant kidney cancer. Exemplarily, such antineoplastic agent may be selected from the group consisting of chemotherapeutics, hormones and analogue thereof and other antineoplastic agent.

Exemplarily, such antineoplastic agent may be selected from the group consisting of platins (e.g., cisplatin, carboplatin, oxaliplatin), anti-metabolites (e.g., azathioprine, 6-mercaptopurine, mercaptopurine, 5-fluorouracil, pyrimidines, thioguanine, fludarabine, floxuridine, cytosine arabinoside (cytarabine), pemetrexed, raltitrexed, pralatrexate, methotrexate), further alkylating agents (e.g., chlorambucil, Ifosfamide mechlorethamine, cyclophosphamide), statins (e.g., cerivastatin, simvastatin, lovastatin, somatostatin, fluvastatin, nystatin, rosuvastatin, atorvastatin, pravastatin, pitavastatin, pentostatin), terpenoids and plant alkaloids (e.g., vinca alkaloids (vincristine, vinblastine, vinorelbine, vindesine), taxanes (e.g., paclitaxel), cytoxan), topoisomerase inhibitors (e.g., camptothecins: irinotecan, topotecan, etoposide, etoposide phosphate, teniposide), melphalan, other antineoplastica (e.g., doxorubicin (adriamycin), doxorubicin lipo, epirubicin, bleomycin)), actinomycin D, aminoglutethimide, amsacrine, anastrozole, antagonists of purine and pyrimidine bases, anthracyclines, aromatase inhibitors, asparaginase, antiestrogens, bexarotene, buserelin, busulfan, camptothecin derivatives, capecitabine, carmustine, cladribine, cytarabine, cytosine arabinoside, alkylating cytostatics, dacarbazine, daunorubicin, docetaxel, epirubicin, estramustine, etoposide, exemestane, fludarabine, fluorouracil, folic acid antagonists, formestane, gemcitabine, glucocorticoids, goserelin, hormones and hormone antagonists, hycamtin, hydroxyurea, idarubicin, irinotecan, letrozole, leuprorelin, lomustine, mercaptopurine, miltefosine, mitomycins, mitosis inhibitors, mitoxantrone, nimustine, procarbazine, tamoxifen, temozolomide, teniposide, testolactone, thiotepa, topoisomerase inhibitors, treosulfan, tretinoin, triptorelin, trofosfamide, cytostatically active antibiotics, everolimus, pimecrolimus, tacrolimus, azithromycin, spiramycin, sirolimus (rapamycin), roxithromycin, ascomycin, bafilomycin, erythromycin, midecamycin, josamycin, concancamycin, clarithromycin, troleandomycin, folimycin, tobramycin, mutamycin, dactinomycin, dactinomycin, rebeccamycin, 4-hydroxyoxycyclophosphamide, bendamustine, thymosin α-1, aclarubicin, fludarabine-5′-dihydrogen phosphate, hydroxycarbamide, aldesleukin, pegaspargase, cepharanthine, epothilone A and B, azathioprine, mycophenolate mofetil, c-myc antisense, b-myc antisense, betulinic acid, camptothecin, melanocyte stimulating hormone (α-MSH), activated protein C, IL-1β inhibitor, fumaric acid and esters thereof, dermicidin, calcipotriol, taclacitol, lapachol, β-lapachone, podophyllotoxin, betulin, podophyllic acid 2-ethyl hydrazide, sagramostim, (rhuGM-CSF), peginterferon α-2b, lenograstim (r-HuG-CSF), filgrastim, macrogol, cephalomannine, selectin (cytokine antagonist), CETP inhibitor, cadherins, cytokinin inhibitors, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, COX inhibitor (e.g., COX-2 and/or COX-3 inhibitor), angiopeptin, ciprofloxacin, fluroblastin, bFGF antagonists, probucol, prostaglandins, 1,11-dimethoxyeanthin-6-one, 1-hydroxy-11-methoxycanthin-6-one, scopoletin, colchicine, NO donors, pentaerythrityl tetranitrate, sydnonimines, S-nitroso derivatives, staurosporine, β-estradiol, α-estradiol, estriol, estrone, ethinyl estradiol, fosfestrol, medroxyprogesterone, estradiol cypionates, cudraisoflavone A, curcumin, dihydronitidine, nitidine chloride, 12-beta-hydroxypregnadiene-3,20-dione bilobol, ginkgol, ginkgolic acid, helenalin, indicine, indicine-N-oxide, lasiocarpine, inotodiol, glycoside 1a, justicidin A and B, larreatin, malloterin, mallotochromanol, isobutyrylmallotochromanol, marchantin A, maytansine, lycoridicin, margetine, pancratistatin, liriodenine, bisparthenolidine, oxoushinsunine, aristolactam-AII, estradiot benzoates, tranilast, kamebakaurin, verapamil, ciclosporin A, paclitaxel and derivatives thereof such as 6-α-hydroxy paclitaxel, baccatin, taxotere, mofebutazone, acemetacin, diclofenac, Ionazolac, dapsone, o-carbamoyl-phenoxy-acetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterol, ademetionine, myrtecaine, polidocanol, nonivamide, levomenthol, benzocaine, aescin, elipticine, D-24851 (Calbiochem), colcemid, cytochalasin A-E, indanocine, nocodazole, bacitracin, vitronectin receptor antagonists, azelastine, free nucleic acids, nucleic acids incorporated into virus transmitters, DNA and RNA fragments, plasminogen activator inhibitor-1, plasminogen activator inhibitor-2, antisense oligonucleotide, VEGF inhibitors, IGF-1, active agents from the group of antibiotics such as cefadroxil, cefazolin, cefaclor, cefoxitin, gentamicin, penicillins, dicloxacillin, oxacillin, sulfonamides, metronidazole, antithrombotics, argatroban, aspirin, abciximab, synthetic antithrombin, bivalirudin, coumadin, enoxaparin, GpIIb/IIIa platelet membrane receptor, antibodies to factor Xa inhibitor, heparin, hirudin, r-hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, NF-kB, Bcl-xL antisense oligonucleotides, halofuginone, nifedipine, tocopherol, molsidomine, tea polyphenols, epicatechin gallate, epigallocatechin gallate, boswellic acids and derivatives thereof, leflunomide, anakinra, etanercept, sulfasalazine, tetracycline, triamcinolone, procainimide, retinoic acid, quinidine, disopyramide, flecainide, propafenone, sotalol, amiodarone, natural and synthetically obtained steroids such as bryophyllin A, inotodiol, maquiroside A, mansonine, strebloside, hydrocortisone, betamethasone, dexamethasone, fenoprofen, ibuprofen, indomethacin, naproxen, phenylbutazone, acyclovir, ganciclovir, zidovudine, antimycotics, clotrimazole, flucytosine, griseofulvin, ketoconazole, miconazole, terbinafine, chloroquine, mefloquine, quinine, natural terpenoids, hippocaesculin, barringtogenol-C₂₁-angelate 14-dehydroagrostistachin, agroskerin, hyptatic acid A, zeorin, strychnophylline, usambarine, usambarensine, daphnoretin, lariciresinol, methoxylariciresinol, syringaresinol, umbelliferone, afromoson, acetylvismione B, desacetylvismione A, vismione A and B, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrine, taxamairin A and B, regenilol, triptolide, cymarin, apocymarin, aristolochic acid, anopterin, hydroxyanopterin, anemonin, protoanemonin, berberine, cheliburin chloride, cicutoxin, sinococuline, combrestatin A and B, periplocoside A, ghalakinoside, deoxypsorospermin, psychorubin, ricin A, sanguinarine, manwu wheat acid, methylsorbifolin, chromones of spathelia, stizophyllin, akagerine, dihydrousambaraensine, hydroxyusambarine, strychnopentamine, Bosentan, BQ123 and BQ788 a pharmaceutically acceptable salt of any thereof, and a combination of two or more thereof or two or more pharmaceutically acceptable salts thereof.

An antineoplastic agent may also be an agent suitable for immunotherapy of neoplasia. An agent suitable for immunotherapy of neoplasia may be understood in the broadest sense as any agent suitable to stimulate the immune system to treat neoplasia. It may be active, passive or a mixture of both (hybrid). In this context, immunotherapy may base on the detectability of neoplasm-associated antigens (often also designated as tumour-associated antigens (TAAs)). Active immunotherapy may direct the immune system to attack neoplastic cells by targeting neoplasm-associated antigens. Passive immunotherapies may enhance existing antineoplastic responses and include the use of antibodies or fragments or variants thereof, immune cells (e.g., lymphocytes (e.g., T-lymphocytes, B-lymphocytes), natural killer cells, lymphokine-activated killer cells, cytokine-activated killer cells, cytotoxic T cells and dendritic cells) and/or cytokines, in particular (optionally humanized) monoclonal antibodies or fragments thereof. Depending on the individual setup, such antibodies or fragments or variants thereof, immune cells and/or cytokines may lead to antibody-dependent cell-mediated cytotoxicity, may activate the complement system, and/or may prevent a receptor from interacting with its ligand. Thereby, in some setups, the targeted cell may be triggered into apoptosis. Examples for antibodies usable in the context of immune therapy include alemtuzumab, ipilimumab, nivolumab, ofatumumab and rituximab. Antibodies or fragments or variants thereof may optionally also be conjugated (e.g., by a radioactive ion). Additionally or alternatively, also dendritic cell therapy may be used. Additionally or alternatively, also cytokines, keyhole limpet hemocyanin, Freund's adjuvant, Bacillus Calmette-Guérin (BCG) vaccine and/or peginterferon alfa-2a may be used. Alternatively or additionally, also an antineoplastic vaccine may be used such as, e.g., a vaccine made of neoplastic tissue or an artificial vaccine (e.g., polypeptide-based, polynucleotide-based, glycoside-based, etc.). The person skilled in the art will be aware of several further agents suitable for immunotherapy of neoplasia usable in the context of the present invention.

Alternatively or additionally, treating may also be or comprise surgical means of excising the neoplastic tissue, in particular malignant kidney cancer tissue, from the individual's body. This may optionally also be the excision (also: resection or enucleation) of a kidney.

Alternatively or additionally, treating may also be or comprise radiation therapy suitable for treating neoplastic tissue in the kidney, in particular malignant kidney cancer tissue. Radiation therapy may exemplarily be or comprise irradiation with x-ray radiation, ultraviolet (UV) radiation (e.g., UV-A, UV-B, and/or UV-C radiation), alpha radiation, beta radiation, gamma radiation, or cosmic radiation. Radiation therapy may also include, but may not be limited to Intensity-Modulated Radiation Therapy (IMRT), 3-Dimensional Conformal Radiotherapy (3DCRT), Stereotactic body radiation therapy (SBRT), Stereotactic radiosurgery (SRS), image-guided radiation therapy (IGRT), Particle Therapy (e.g, proton therapy), Brachytherapy, Radioisotope Therapy (RIT) (e.g., with iodine-131, lutetium-177, strontium-89 and samarium (153Sm) lexidronam and/or yttrium-90).

A further aspect of the present invention relates to a method for detecting malignant kidney cancer in an individual, said method conducted in vitro comprising the following steps:

(i) providing a urine sample U obtained from the individual; and

(ii) determining Vim3 level in the urine sample U.

It will be understood that the specifications made in the context of the method of detecting Mxi-2 as described above apply mutatis mutandis to the method of detecting Vim3 instead, except that the presence, in particular an increased level, of Vim3 indicates a benign neoplasia, in particular a benign (kidney) oncocytoma. In other words, in particular when there is a single neoplasia in the individual of interest, the presence, in particular an increased level, of Vim3 is a hint for the absence of malignant kidney cancer in the individual. Worded the other way round, a decreased Vim3 level, in particular the absence of Vim3, in neoplastic tissue or body fluids contacted with such tissue indicates the presence of malignant kidney cancer in the individual.

As mentioned above, the present invention relates to a method for detecting malignant kidney cancer and/or a (benign kidney) oncocytoma in an individual, said method conducted in vitro, comprising determining one or more biomarker levels selected from the group consisting of the Mxi-2 level, the Vim3 level, the MAPK p38 level and the Atg7 level in a body fluid sample, in particular a urine sample U, from the individual.

Accordingly, the present invention relates to a method for detecting malignant kidney cancer and/or a (benign kidney) oncocytoma in an individual, said method conducted in vitro, comprising determining at least one of the following levels or combination or levels in a body fluid sample, in particular a urine sample U, from the individual:

(i) Mxi-2 level; (ii) Vim3 level; (iii) MAPK p38 level; (iv) Atg7 level; (v) Mxi-2 and Vim3 level; (vi) Mxi-2 and MAPK p38 level; (vii) Vim3 and Atg7 level; (viii) Vim3 and MAPK p38 level; (ix) MAPK p38 and Atg7 level; (x) Mxi-2, Vim3 and Atg7 level; (xi) Mxi-2, Vim3 and MAPK p38 level; (xii) Atg7, Vim3 and MAPK p38 level; (xiii) Mxi-2, Atg7 and MAPK p38 level; and (xiv) Mxi-2, Vim3, Atg7 and MAPK p38 level.

It will be understood that the preferred embodiments defined in the context of Mxi-2 herein, mutatis mutandis apply to this method.

In a preferred embodiment, an increased level selected from the group consisting of Mxi-2, MAPK p38 and Atg7 indicates the presence of malignant kidney cancer in the individual, in particular wherein the malignant kidney cancer is selected from the group consisting of RCC, chromo RCC, pap RCC and/or eosino RCC. In a preferred embodiment, an increased Vim3 level indicates the presence of a (benign kidney) oncocytoma in the individual.

As used herein, an increase of a level (e.g., of any of Mxi-2, MAPK p38, Atg7 and/or Vim3) is each independently from another preferably an increase by at least 5%, in particular by at least 10%, at least 15% or at least 20% or more, in comparison to a control sample C as indicated above, i.e., a comparable sample from a healthy individual, in other words a sample obtained from a control individual of the same species free of malignant cancer cells and free of an (benign kidney) oncocytoma. In a preferred embodiment, the level of any of Mxi-2, MAPK p38, Atg7 and/or Vim3 in a body fluid, in particular in a urine sample U, may be independently from another in a range of from 1 pg (pictogram) to 1000 ng (nanogram) per 100 ml, of from 10 pg to 500 ng per 100 ml, of from 20 pg to 250 ng per 100 ml, of from 20 pg to 100 ng per 100 ml, of from 50 pg to 90 ng per 100 ml, of from 60 pg to 100 pg per 100 ml, of from 80 pg to 500 pg per 100 ml, of from 100 pg to 750 pg per 100 ml, of from 150 pg to 1 ng per 100 ml, of from 0.5 ng to 2 ng per 100 ml, of from 1 ng to 5 ng per 100 ml, of from 2 ng to 10 ng per 100 ml, of from 5 ng to 20 ng per 100 ml, of from 10 ng to 50 ng per 100 ml, or of from 25 ng to 10 ng per 100 ml.

The present invention further relates to a method for detecting (also: diagnosing) malignant kidney cancer in an individual, comprising determining Vim3 level in a body fluid, in particular a urine sample U, of an individual.

The present invention further relates to a method for detecting an (benign kidney) oncocytoma (also: diagnosing an (benign kidney) oncocytoma) in an individual, comprising determining Vim3 level in a body fluid, in particular a urine sample U, of an individual.

The present invention further relates to a method for detecting (also: diagnosing) malignant kidney cancer in an individual, comprising determining Mxi-2 level in a body fluid, in particular a urine sample U, of an individual.

The present invention further relates to a method for detecting an (benign kidney) oncocytoma (also: diagnosing an (benign kidney) oncocytoma) in an individual, comprising determining Mxi-2 level in a body fluid, in particular a urine sample U, of an individual.

The present invention further relates to a method for detecting (also: diagnosing) malignant kidney cancer in an individual, comprising determining Vim3 and Mxi-2 level in a body fluid, in particular a urine sample U, of an individual.

The present invention further relates to a method for detecting an (benign kidney) oncocytoma (also: diagnosing an (benign kidney) oncocytoma) in an individual, comprising determining Vim3 and Mxi-2 level in a body fluid, in particular a urine sample U, of an individual.

An aspect of the present invention relates to a method for detecting an (benign kidney) oncocytoma (also: diagnosing an (benign kidney) kidney oncocytoma) in an individual, said method (preferably conducted in vitro) comprising the following steps:

(i) providing a urine sample U obtained from the individual; and

(ii) determining Mxi-2 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Vim3 level in the urine sample U.

In other words, the present invention further relates to a method for diagnosing an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Vim3 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Mxi-2 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Vim3 and Mxi-2 level in the urine sample U.

The conclusions of the determined Mxi-2 and/or Vim3 levels may be as described herein, in particular as described above. It will be understood that the properties of a patient, malignant kidney cancer, (benign kidney) oncocytoma, sample, etc., are preferably as defined herein. The above definitions and preferred embodiments apply mutatis mutandis.

The present invention further relates to a method for detecting (also: diagnosing) malignant kidney cancer in an individual, comprising determining Atg7 level in a body fluid, in particular a urine sample U, of an individual.

In a preferred embodiment, an increased Atg7 level indicates the presence of malignant kidney cancer in the individual, in particular wherein the malignant kidney cancer is selected from the group consisting of RCC, chromo RCC and/or eosino RCC.

The present invention further relates to a method for detecting an (benign kidney) oncocytoma (also: diagnosing an (benign kidney) oncocytoma) in an individual, comprising determining Atg7 level in a body fluid, in particular a urine sample U, of an individual.

The present invention further relates to a method for detecting (also: diagnosing) malignant kidney cancer in an individual, comprising determining Atg7 and Mxi-2 level in a body fluid, in particular a urine sample U, of an individual.

In a preferred embodiment, increased levels of Mxi-2 and Atg7 indicate the presence of malignant kidney cancer in the individual, in particular wherein the malignant kidney cancer is selected from the group consisting of RCC, chromo RCC, pap RCC and/or eosino RCC.

The present invention further relates to a method for detecting an (benign kidney) oncocytoma (also: diagnosing an (benign kidney) oncocytoma) in an individual, comprising determining Atg7 and Mxi-2 level in a body fluid, in particular a urine sample U, of an individual.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Atg7 level in the urine sample U.

In other words, the present invention further relates to a method for diagnosing an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Atg7 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Mxi-2 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Atg7 and/or Mxi-2 level in the urine sample U.

The conclusions of the determined Mxi-2 and/or Atg7 levels may be as described herein, in particular as described above. It will be understood that the properties of a patient, malignant kidney cancer, (benign kidney) oncocytoma, sample, etc., are preferably as defined herein. The above definitions and preferred embodiments apply mutatis mutandis.

The present invention further relates to a method for detecting (also: diagnosing) malignant kidney cancer in an individual, comprising determining MAPK p38 level in a body fluid, in particular a urine sample U, of an individual.

In a preferred embodiment, an increased MAPK p38 level indicates the presence of malignant kidney cancer in the individual, in particular wherein the malignant kidney cancer is selected from the group consisting of RCC, chromo RCC and/or eosino RCC.

The present invention further relates to a method for detecting an (benign kidney) oncocytoma (also: diagnosing an (benign kidney) oncocytoma) in an individual, comprising determining MAPK p38 level in a body fluid, in particular a urine sample U, of an individual.

The present invention further relates to a method for detecting (also: diagnosing) malignant kidney cancer in an individual, comprising determining MAPK p38 and Mxi-2 level in a body fluid, in particular a urine sample U, of an individual.

In a preferred embodiment, increased levels of Mxi-2 and MAPK p38 indicate the presence of malignant kidney cancer in the individual, in particular wherein the malignant kidney cancer is selected from the group consisting of RCC, chromo RCC, pap RCC and/or eosino RCC.

In a preferred embodiment, increased levels of Mxi-2, Atg7 and MAPK p38 indicate the presence of malignant kidney cancer in the individual, in particular wherein the malignant kidney cancer is selected from the group consisting of RCC, chromo RCC, pap RCC and/or eosino RCC.

The present invention further relates to a method for detecting an (benign kidney) oncocytoma (also: diagnosing an (benign kidney) oncocytoma) in an individual, comprising determining MAPK p38 and Mxi-2 level in a body fluid, in particular a urine sample U, of an individual.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining MAPK p38 level in the urine sample U.

In other words, the present invention further relates to a method for diagnosing an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining MAPK p38 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining Mxi-2 level in the urine sample U.

An aspect of the present invention relates to a method for detecting an (benign kidney) oncocytoma (also: diagnosing an (benign kidney) oncocytoma) in an individual, said method (preferably conducted in vitro) comprising the following steps:

(i) providing a urine sample U obtained from the individual; and

(ii) determining MAPK p38 and Mxi-2 level in the urine sample U.

The conclusions of the determined Mxi-2 and/or MAPK p38 levels may be as described herein, in particular as described above. It will be understood that the properties of a patient, malignant kidney cancer, (benign kidney) oncocytoma, sample, etc., are preferably as defined herein. The above definitions and preferred embodiments apply mutatis mutandis.

Another aspect of the present invention relates to a method for detecting an (benign kidney) oncocytoma in an individual, said method conducted in vitro, comprising determining the Vim3 level in a urine sample U from the individual.

A still further aspect of the present invention relates to a method for detecting an malignant kidney cancer in an individual, said method conducted in vitro, comprising determining the Vim3 level in a urine sample U from the individual.

In a preferred embodiment, the method for detecting an (benign kidney) oncocytoma in an individual, said method conducted in vitro, comprises the following steps:

(i) providing a urine sample U obtained from the individual; and

(ii) determining Vim3 level in the urine sample U.

In a preferred embodiment, the method for detecting an (benign kidney) oncocytoma in an individual, said method conducted in vitro, comprises the step of

(iii) comparing the Vim3 level determined in step (ii) with
(a) a predetermined reference value R1 indicating the borderline between a sample indicating the presence of a (benign kidney) oncocytoma and a sample indicating the absence of malignant kidney cancer; and/or
(b) Vim3 level determined in a control sample C obtained from a control individual of the same species free of an (benign kidney) oncocytoma,

wherein an Vim3 level determined in the urine sample U that is higher than R1 and/or at least 20% higher than the Vim3 level of C indicates the presence of an (benign kidney) oncocytoma in the individual, wherein the Vim3 level in each case is related to the total polypeptide content comprised in the respective sample.

As already laid out above, all specifications, aspects and embodiments described in the context of the urine sample U may apply mutatis mutandis to any body fluid sample in general. This applies particularly to those body fluids that may potentially comprise Mxi-2 and/or Vim3, in particularly such body fluid that may physiologically et in contact with the kidney. Thus, also a method for detecting malignant kidney cancer in an individual is disclosed, wherein said method is conducted in vitro comprising the following steps:

(i) providing a body fluid sample obtained from the individual; and

(ii) determining Vim3 level in the sample.

It will be understand that means for conducting the method of the present invention may also be compiled in a useful package unit.

Accordingly, a further aspect of the present invention relates to a kit for use in a method according to the present invention, comprising:

(A) means for determining the Mxi-2 level in an urine sample U;

(B) means for determining the Vim3 level in an urine sample U; and

(C) instructions for carrying out the method according to the present invention.

It will be understood that the specifications made in the context of the method as described above apply mutatis mutandis to the kit of the present invention. As already laid out above, all specifications, aspects and embodiments described in the context of the urine sample U may apply mutatis mutandis to any body fluid sample in general. This applies particularly to those body fluids that may potentially comprise Mxi-2 and/or Vim3, in particularly such body fluid that may physiologically get in contact with the kidney. Thus, also a kit for use in a method according to the present invention is disclosed that comprises:

(A) means for determining the Mxi-2 level in a body fluid sample;

(B) means for determining the Vim3 level in a body fluid sample; and

(C) instructions for carrying out the method according to the present invention.

A kit generally includes a package with one or more containers holding the reagents, as one or more separate compositions or, optionally, as a mixture if reagents are compatible. The kit may also include other material(s), which may be desirable from a user standpoint, such as a buffer(s), a diluent(s), a standard(s), and/or any other material useful in sample processing, washing, or conducting any other step of the assay.

Means for determining the Mxi-2 level in a urine sample U may be any means suitable for this purpose. It is referred to the method of the present invention as laid out herein. In particular, such means may comprise Mxi-2-specific antibodies or variants or fragments thereof, and optionally secondary antibodies. Such antibodies or variants or fragments thereof, and optionally secondary antibodies may optionally be immobilized and/or labelled. This is described in more detail below. Preferably, such means are suitable for detecting the Mxi-2 polypeptide. Alternatively or additionally, such means may comprise primers (typically a forward and a reverse primer) for amplifying the Mxi-2 mRNA.

Mxi-2 mRNA may be also detected as described in U.S. Pat. No. 6,017,692, in particular by means of PCR techniques based on the primers described therein.

Means for determining the Vim3 level in a urine sample U may be any means suitable for this purpose. It is referred to the method of the present invention as laid out herein. In particular, such means may comprise Vim3-specific antibodies or variants or fragments thereof, and optionally secondary antibodies. Such antibodies or variants or fragments thereof, and optionally secondary antibodies may optionally be immobilized and/or labelled. This is described in more detail below. Preferably, such means are suitable for detecting the Vim3 polypeptide. Alternatively or additionally, such means may comprise primers (typically a forward and a reverse primer) for amplifying the Vim3 mRNA.

In a particularly preferred embodiment, the means for determining the Mxi-2 level (and optionally also the Vim3 level) form part of a dipstick. This is described in more detail below.

A kit according to the present invention may include a solid phase and a capture agent affixed to the solid phase, wherein the capture agent is an antibody specific for the analysis of a urine sample U (e.g., at least one Mxi-2 and/or a Vim3-specific antibody, in particular both each at least one Mxi-2 and at least one Vim3-specific antibody). The solid phase may comprise a material such as a magnetic or paramagnetic particle including a microparticle, a bead, a test tube, a microtiter plate, a cuvette, a membrane, a scaffolding molecule, a quartz crystal, a film, a filter paper, a dipstick a disc or a chip.

Furthermore, a kit according to the present invention may preferably further comprise user instructions for carrying out the method of the present invention. Instructions included in kits of the invention may be affixed to packaging material or may be included as a package insert. While the instructions are typically written or printed materials they are not limited to such. Any medium capable of storing such instructions and communicating them to an end user is contemplated by this invention. Such media include, for example, computer media including, but are not limited to, electronic storage media (e.g., magnetic discs, tapes, cartridges, chips), optical media (e.g., CD ROM), and the like.

As indicated above, the method of the present invention can also be conducted by means of a dipstick analysis (lateral flow analysis).

Accordingly, a still further aspect of the present invention refers to a dipstick usable for the method of the present invention comprising, placed in the direction of flow of the urine sample U, on a carrier that is suitable for soaking the urine sample U, the following:
(0) an edge or segment suitable for soaking the urine sample U;
(1) optionally a stripe (1) comprising labeled Mxi-2-specific antibodies or antibody fragments which are not immobilized and freely movable when the urine sample U passes through this stripe (1);
(2) a stripe (2) comprising immobilized unlabeled MAPK p38-specific, in particular Mxi-2-specific, antibodies or antibody fragments; and
(3) optionally a stripe (3) of immobilized unlabeled antibodies or antibody fragments specifically binding the labeled Mxi-2-specific antibodies or antibody fragments of stripe (1).

It will be understood that the specifications made in the context of the method and kit as described above apply mutatis mutandis to the dipstick of the present invention. As already laid out above, all specifications, aspects and embodiments described in the context of the urine sample U may apply mutatis mutandis to any body fluid sample in general. This applies particularly to those body fluids that may potentially comprise Mxi-2 and/or Vim3, in particularly such body fluid that may physiologically get in contact with the kidney. Thus, also a dipstick usable for the method of the present invention comprising, placed in the direction of flow of the body fluid sample, on a carrier that is suitable for soaking said sample, the following:
(0) an edge or segment suitable for soaking the body fluid sample;
(1) optionally a stripe (1) comprising labeled Mxi-2-specific antibodies or antibody fragments which are not immobilized and freely movable when the body fluid sample passes through this stripe (1);
(2) a stripe (2) comprising immobilized unlabeled MAPK p38-specific, in particular Mxi-2-specific, antibodies or antibody fragments; and
(3) optionally a stripe (3) of immobilized unlabeled antibodies or antibody fragments specifically binding the labeled Mxi-2-specific antibodies or antibody fragments of stripe (1).

Accordingly, a dipstick according to the present invention (preferably usable for the method of or the use of the present invention) comprise at least, placed in the direction of flow of the urine sample U, on a carrier that is suitable for soaking the urine sample U, the following:
(0) an edge or segment suitable for soaking the urine sample U;
(2) a stripe (2) comprising immobilized unlabeled MAPK p38-specific, in particular Mxi-2-specific, antibodies or antibody fragments.

Examples for setups are provided in FIGS. 2, 3 and 4 herein.

As used herein, the terms “dipstick”, “dip-stick”, “test strip”, “control strip”, “diagnostic/medical dipstick” may be understood interchangeably in the broadest sense as any device that is usable to test a urine sample U in the context of the present invention (according to the lateral flow technique).

In the context of the dipstick, the urine sample U is typically liquid, semi-liquid or liquefied so that it can be soaked by a carrier of the dipstick. Typically, the urine sample U comprises an aqueous liquid. Exemplarily, the urine sample U usable by the dipstick may be urine from an individual.

In particular if the dipstick lacks stripe (1), the urine sample U is preferably premixed with a labeled Mxi-2-specific antibody or antibody fragment or fragment thereof. The volume and molar ratios will be adapted accordingly in order to optimize binding efficiency.

The volume of the urine sample U (optionally diluted and/or premixed with a labeled Mxi-2-specific antibody or antibody fragment) added to the dipstick will be adapted to the size and material of the dipstick. Typical volumes for adding to a segment suitable for soaking the urine sample U are in the range of from 10 to 1000 μl, preferably 50 to 500 μl, in particular 75 to 300 μl, exemplarily (approximately 200 μl).

Exemplarily, the carrier may be a (hydro) gel or a piece of paper board, and may be optionally film laminated. Typically, the dipstick will be stored in dry state and is moistened by the urine sample U. When conducting the method of the present invention by means of the dipstick, the edge or segment suitable for soaking the urine sample U (0) may be contacted with the urine sample U. This is preferably conducted long enough to enable the sample liquid to be soaked in the carrier of the dipstick. The other parts of the dipstick are preferably not directly contacted with the urine sample U.

It is preferably enabled that the urine sample U flows through the carrier of the dipstick at least until the stripes (1) (if present) and (2) and optionally (3) have been passed by the urine sample U or parts thereof.

According to a preferred embodiment, the urine sample U is of a first species and the antibodies or antibody fragments of each of stripe (1) (if present) or the antibodies or fragments used for premixing with the urine sample U (in particular if stripe (1) is not present) on the one hand and (2) and optionally (3) of the other hand are each of different species.

In a preferred embodiment, the immobilized unlabeled antibodies or antibody fragments of stripe (3) specifically bind to the Fc fragment of the labeled Mxi-2-specific antibodies or antibody fragments of stripe (1) (if present) or premixed with the optionally diluted urine (in particular if stripe (1) is not present). Exemplarily, the Mxi-2-specific antibodies or antibody fragments which are not immobilized are (preferably monoclonal) rabbit antibodies. Then, the immobilized antibodies of stripe (3) may be (preferably monoclonal) antibodies directed against the Fc part of the antibodies provided in stripe (1) or premixed with the urine sample and optionally one or more buffers (in particular if stripe (1) is not present).

The label may be a fluorescence label, a visible dye label or, particularly preferably, a (colloidal) gold label. Such (colloidal) gold may be added to an antibody or antibody fragment bay any means, exemplarily by means of a GOLD Conjugation Kit.

When a Mxi-2-containing sample of an individual bearing malignant kidney cancer is added to the dipstick, upon flowing through the dipstick, the labeled Mxi-2-specific antibodies may bind to Mxi-2 in the urine sample U, thereby forming a Mxi-2/antibody conjugate. This conjugate will then binding to the unlabeled Mxi-2-specific antibodies of stripe.

When a sample lacking Mxi-2 of a healthy individual is added to the dipstick, upon flowing through the dipstick, the labeled Mxi-2-specific antibodies will not form a Mxi-2/antibody conjugate. Therefore, the polypeptides comprised in the urine sample U will then pass by the stripe (2) without being bound and will pass through the dipstick until the stripe (3).

In such dipstick, the ratio between signal intensity of the label in stripe (2) and (3) indicates the presence or absence of malignant kidney cancer in the individual the urine sample U has been obtained from. A higher (2):(3) ratio indicates higher probability of the presence of kidney cancer in the individual, whereas a lower (2):(3) ratio indicates lower probability of the presence of kidney cancer in the individual in the sense of the method of the present invention laid out above.

In a preferred embodiment, the dipstick of the present invention comprises, placed in the direction of flow of the urine sample U, on a carrier that is suitable for soaking the urine sample U, the following:
(0) an edge or segment suitable for soaking the urine sample U;
(1) a stripe (1) comprising labeled Mxi-2-specific antibodies or antibody fragments which are not immobilized and freely movable when the urine sample U passes through this stripe (1);
(2) a stripe (2) comprising immobilized unlabeled MAPK p38-specific, in particular Mxi-2-specific, antibodies or antibody fragments; and
(3) optionally a stripe (3) of immobilized unlabeled antibodies or antibody fragments specifically binding the labeled Mxi-2-specific antibodies or antibody fragments of stripe (1).

In an alternative preferred embodiment, the dipstick (preferably usable for the method of or the use of the present invention) comprises, placed in the direction of flow of the urine sample U premixed with labeled Mxi-2-specific antibodies or variants or antibody fragments thereof (which are not immobilized and freely movable) on a carrier that is suitable for soaking the urine sample U premixed with labeled Mxi-2-specific antibodies or antibody fragments, the following:
(0) an edge or segment suitable for soaking the urine sample U;
(2) a stripe (2) comprising immobilized unlabeled MAPK p38-specific, in particular Mxi-2-specific, antibodies or antibody fragments; and
(3) a stripe (3) of immobilized unlabeled antibodies or antibody fragments specifically binding the labeled Mxi-2-specific antibodies or antibody fragments of stripe (1).

The premixing of the labeled Mxi-2-specific antibodies or antibody fragments may be followed by an incubation to allow and optimize binding of the Mxi-2-specific antibodies or antibody fragments to its molecular target Mxi-2. This may exemplarily be performed by incubating for 10 to 120 min, 10 to 60 min or over night, at a temperature of from 2 to 25° C.

Alternatively or additionally, a dipstick according to the present invention may be may be prepared according to Preechakasedkit et al., 2012, Biosens Bioelectron 31(1):562-566; Tao et al., 2014, Lett Appl Microbiol 59(2):247-251 or Wang et al., 2010, J Virol Methods 2010, 170(1-2):80-85.

In the view of the method of the present invention, the dipstick can also be such that it concomitantly detects the presence and preferably also assesses the quantity, of Mxi-2 and Vim3.

Accordingly, in a preferred embodiment, a dipstick of the present invention comprises, placed in the direction of flow of the urine sample U, on a carrier that is suitable for soaking the urine sample U, the following:
(0) an edge or segment suitable for soaking the urine sample U;
(1) optionally a stripe (1) comprising labeled Mxi-2-specific antibodies or antibody fragments which are not immobilized and freely movable when the urine sample U passes through this stripe (1);
(1′) optionally a stripe (1′) comprising labeled Vim3-specific antibodies or antibody fragments which are not immobilized and freely movable when the urine sample U passes through the one or more stripe(s) (1);
(2) a stripe (2) comprising immobilized unlabeled MAPK p38-specific, in particular Mxi-2-specific, antibodies or antibody fragments;
(2′) a stripe (2′) comprising immobilized unlabeled Vimentin-specific, in particular Vim3-specific, antibodies or antibody fragments;
(3) optionally a stripe (3) of immobilized unlabeled antibodies or antibody fragments specifically binding the labeled Mxi-2-specific antibodies or antibody fragments of stripe (1); and
(3′) optionally a stripe (3′) of immobilized unlabeled antibodies or antibody fragments specifically binding the labeled Vim3-specific antibodies or antibody fragments of stripe (1).

Such dipstick enables the concomitant (semi)quantitative detection of Mxi-2 and Vim3. This enables also to calculate the ration of a Mxi-2:Vim3 ratio as described above.

The stripes (1′), (2′) and (2′) are mutatis mutandis defined as the corresponding stripes (1), (2) and (2) as described above except that Mxi-2 is replaced by Vim3 and, accordingly, the Mxi-2-specific antibody or variant or fragment thereof is replaced by a Vim3-specific antibody or variant or fragment thereof.

The stripes (3) and (3′) may also form a single stripe. Then stripe (3) may comprise immobilized unlabeled antibodies concomitantly binding the labeled Mxi-2-specific and Vim3-specific antibodies (depicted as stars and x), in particular when these have the same or a comparable Fc fragment. It will be understood that the stripes (1) and (1′), as far as present, may be present as locally separated stripes or may form a single stripe comprising a mixture containing Mxi-2-specific antibodies and Vim3-specific antibodies.

In a particularly preferred embodiment, the dipstick of the present invention comprises, placed in the direction of flow of the urine sample U, on a carrier that is suitable for soaking the urine sample U, the following:
(0) an edge or segment suitable for soaking the urine sample U;
(1) optionally a stripe (1) comprising labeled Mxi-2-specific antibodies or antibody fragments which are not immobilized and freely movable when the urine sample U passes through this stripe (1);
(1′) optionally a stripe (1′) comprising labeled Vim3-specific antibodies or antibody fragments which are not immobilized and freely movable when the urine sample U passes through the one or more stripe(s) (1);
(2) a stripe (2) comprising immobilized unlabeled MAPK p38-specific, in particular Mxi-2-specific, antibodies or antibody fragments;
(2′) a stripe (2′) comprising immobilized unlabeled Vimentin-specific, in particular Vim3-specific, antibodies or antibody fragments;
(3) a stripe (3) of immobilized unlabeled antibodies or antibody fragments specifically binding the labeled Mxi-2-specific antibodies or antibody fragments of stripe (1); and
(3′) a stripe (3′) of immobilized unlabeled antibodies or antibody fragments specifically binding the labeled Vim3-specific antibodies or antibody fragments of stripe (1).

An example of such dipstick is provided in FIG. 4 herein (wherein (3) and (3′) are indicated as two separate stripes of (3)). The stripes (3) and (3′) may also form a single stripe. Then stripe (3) may comprise immobilized unlabeled antibodies concomitantly binding the labeled Mxi-2-specific and Vim3-specific antibodies (depicted as stars and x), in particular when these have the same or a comparable Fc fragment.

Also the Mxi-2 antibody or fragment or variant thereof of the present invention as such bears special characteristics. Accordingly, a still further aspect of the present invention refers to an antibody or fragment or variant thereof specific for Mxi-2 polypeptide. In a highly preferred embodiment, the antibody or fragment or variant thereof specific for Mxi-2 polypeptide is specifically binds to the epitope of Mxi-2 having the sequence GKLTIYPHLMDIELVMI (SEQ ID NO: 4) or a truncated sequence thereof (e.g., truncated by one, two three, four, five or more amino acid moieties of the N- or the C-terminus). It will be understood that the specifications made in the context of the method, kit and/or dipstick as described above apply mutatis mutandis to the antibody of the present invention.

The antibody or fragment or variant thereof specific for Mxi-2 polypeptide (also anti-Mxi-2 antibody) may be any antibody or fragment or variant thereof specifically binding to Mxi-2.

As used in the context of the present invention, the term “antibody” may be understood in the broadest sense as any type of immunoglobulin or antigen-binding fraction or variant thereof known in the art. Exemplarily, the antibody of the present invention may be an immunoglobulin A (IgA), immunoglobulin D (IgD), immunoglobulin E (IgE), immunoglobulin G (IgG), immunoglobulin M (IgM), immunoglobulin Y (IgY) or immunoglobulin W (IgW). Preferably, the antibody is an IgA, IgG or IgD. More preferably, the antibody is an IgG. However, it will be apparent that the type of antibody may be altered by biotechnological means by cloning the gene encoding for the antigen-binding domains of the antibody of the present invention into a common gene construct encoding for any other antibody type.

The binding between the antibody and its molecular target structure (i.e., its antigen, e.g., Mxi-2) typically is a non-covalent binding. Preferably, the binding affinity of the antibody to its antigen has a dissociation constant (Kd) of less than 1 μM, less than 500 nM, less than 200 nM, less than 100 nM, less than 50 nM, less than 40 nM, less than 30 nM or even less than 20 nM.

Preferably, the binding affinity to Mxi-2 is higher than to full length MAPK p38. In a preferred embodiment, the antibody or fragment or variant thereof binds to the Mxi-2 polypeptide with a dissociation constant of not more than 20 nM and, preferably, to full length MAPK p38 with a dissociation constant of more than 20 nM.

The term “antibody” as used herein may be understood in the broadest sense and also includes what may be designated as an antibody variant (also: antibody mutant). As used in the context of the present invention, the terms “antibody variant” and “antibody mutant” may be understood interchangeably in the broadest sense as any antibody mimetic or antibody with altered sequence known in the art. The antibody variant may have at least 10%, at least 20%, at least 50%, at least 60%, at least 70%, at least 80%, at least 85%, at least 90% or at least 95% of the binding affinity of a corresponding antibody, i.e., bear a dissociation constant (Kd) of less than 10 μM, less than 1 μM, less than 500 nM, less than 200 nM, less than 100 nM, less than 50 nM, less than 40 nM, less than 30 nM or even less than 20 nM.

As used herein, the term “antibody fragment” may be understood in the broadest sense as any fragment of an antibody that still bears binding affinity to its molecular target (i.e., its antigen, e.g., Mxi-2). Exemplarily, the antibody fragment may be a fragment antigen binding (Fab fragment), Fc, F(ab′)₂, Fab′, scFv, a truncated antibody comprising one or both complementarity determining region(s) (CDR(s)) or the variable fragment (Fv) of an antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. either the Fv fragment alone or ‘Fab’-fragments comprising one of the upper arms of the “Y” that includes the Fv plus the first constant domains. These fragments are usually stabilized by introducing a polypeptide link between the two chains which results in the production of a single chain Fv (scFv). Alternatively, disulfide-linked Fv (dsFv) fragments may be used. The binding domains of fragments can be combined with any constant domain in order to produce full length antibodies or can be fused with other polypeptides and polypeptides. A recombinant antibody fragment is the single-chain Fv (scFv) fragment. Dissociation of scFvs results in monomeric scFvs, which can be complexed into dimers (diabodies), trimers (triabodies) or larger aggregates such as TandAbs and Flexibodies. The antibody may be a Fab, a Fab′, a F(ab′)2, a Fv, a disulfide-linked Fv, a scFv, a (scFv)₂, a bivalent antibody, a bispecific antibody, a multispecific antibody, a diabody, a triabody, a tetrabody or a minibody.

As mentioned above, the term “antibody” may also include an antibody mimetic which may be understood in the broadest sense as organic compounds that, like antibodies, can specifically bind antigens and that typically have a molecular mass in a range of from approximately 3 kDa to approximately 25 kDa. Antibody mimetics may be, e.g., affibody molecules (affibodies), aptamers, affilins, affitins, anticalins, avimers, DARPins, Fynomers, Kunitz domain peptides, single-domain antibodies (e.g., VHH antibodies or VNAR antibodies, nanobodies), monobodies, diabodies, triabodies, flexibodies and tandabs. The antibody mimetics may be of natural origin, of gene technologic origin and/or of synthetical origin. The antibody mimetics may also include polynucleotide-based binding units. Optionally, the antibody may also be a CovX-body. Optionally, the antibody may also be a cameloid species antibody.

The antibody according to the present invention is preferably a monoclonal antibody, a chimeric antibody or a humanized antibody. Monoclonal antibodies are monospecific antibodies that are identical because they are produced by one type of immune cell that are all clones of a single parent cell. A chimeric antibody is an antibody in which at least one region of an immunoglobulin of one species is fused to another region of an immunoglobulin of another species by genetic engineering in order to reduce its immunogenicity. For example murine V_{L} and V_{H} regions may be fused to the remaining part of a human immunoglobulin. A particularly preferred type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human.

The antibody or antibody fragment, independent on its chemical nature, may optionally be dissolved in any medium suitable for storing said antibody such as, e.g., water, an aqueous buffer (e.g., a Hepes, Tris, or phosphate buffer (e.g. phosphate buffered saline (PBS)), an organic solvent (e.g., dimethyl sulfoxide (DMSO), dimethylformide (DMF)) or a mixture of two or more thereof. The antibody or variant thereof according to the present invention may be of any species or origin. It may bind to any epitope(s) comprised by its molecular target structure (e.g., linear epitope(s), structural epitope(s), primary epitope(s), secondary epitope(s), e.g., such of Mxi-2). Preferably, the antibody or variant thereof may recognize the naturally folded molecular target structure or a domain or fragment thereof (e.g., Mxi-2 in urine environment). The antibody or variant thereof may be of any origin an antibody may be obtained from such as, e.g., natural origin, a gene technologic origin and/or a synthetic origin. Optionally, the antibody may also be commercially available. The person skilled in the art will understand that the antibody may further comprise one or more posttranscriptional modification(s) and/or may be conjugated to one or more further structures such as label moieties or cell-penetrating peptides (CPPs). Optionally, the antibody or antibody fragment may be added to a support, particularly a solid support such as an array, bead (e.g. glass or magnetic), a fiber, a film etc. The skilled person will be able to adapt the antibody of the present invention and a further component to the intended use by choosing a suitable further component.

Typically, the antibody or fragment or variant thereof specific for Mxi-2 polypeptide of the present invention is obtained from a cell.

Accordingly, a still further aspect of the present invention relates to a cell capable of producing the antibody or fragment or variant thereof according to the present invention.

Such cell may be any cell known in the art for this purpose such as, e.g., a eukaryotic cell (e.g. a mammalian cell (e.g., a human or humanized cell, a mouse cell, a rat cell, a goat cell, a pig cell, a bovine cell, a camel cell, a horse cell, etc.), a bird cell (including bird cells comprised in a bird's egg), a yeast cell, an insect cell) or a bacterial cell. The cell may also be a hybridoma cell as known in the art.

A further aspect of the present invention relates to an antineoplastic agent for use in a method for treating an individual bearing (and optionally suffering from) malignant kidney cancer, wherein the malignant kidney cancer has previously been detected in the individual by means of the method of the present invention. In other words, the present invention also relates to a method for treating an individual bearing (and optionally suffering from) malignant kidney cancer, said method comprising administering the individual with an amount sufficient for treating the malignant kidney cancer, wherein the malignant kidney cancer has previously been detected in the individual by means of the method of the present invention.

It will be understood that all specifications and embodiments described in the context of the method for detecting malignant kidney cancer in an individual according to the invention as laid out above mutatis mutandis apply to this aspect of the invention.

A still further aspect of the present invention relates to a method for treating an individual bearing (and optionally suffering from) a neoplasm, said method comprising:

the steps of the method for detecting malignant kidney cancer in an individual according to the invention, and

the step of treating the neoplasm identified in the preceding step.

In a preferred embodiment, treating the neoplasm is or comprises administering the individual with an amount of an antineoplastic agent sufficient for treating the type of neoplasm identified in the preceding step. Examples for such antineoplastic agent are described above.

In a preferred embodiment, the invention relates to a method for treating an individual bearing (and optionally suffering from) malignant kidney cancer, said method comprising:

the steps of the method for detecting malignant kidney cancer in an individual according to the invention, and

the step of treating the malignant kidney cancer identified in the preceding step.

In a preferred embodiment, treating the malignant kidney cancer is or comprises administering the individual with an amount of an antineoplastic agent sufficient for treating the malignant kidney cancer identified in the preceding step.

Optionally, in case of a benign (kidney) oncocytoma, the individual may also be treated by removal of the kidney or preferably by resection of the benign oncocytoma, then preferably without administering an antineoplastic agent and without irradiation, whereas in case of malignant kidney cancer the individual may preferably be treated by means of administering the individual with an amount of an antineoplastic agent sufficient for treating the malignant kidney cancer identified in the preceding step.

It will be understood that all specifications, aspects and embodiments described in the context of the method for detecting malignant kidney cancer in an individual according to the invention also apply to the method for treating an individual bearing (and optionally suffering from) a neoplasm, in particular malignant kidney cancer.

In a preferred embodiment, the method for treating an individual bearing (optionally suffering from) a neoplasm comprises the following steps:
(i) providing a urine sample U obtained from the individual; and
(ii) determining Mxi-2 and/or Vim3 level(s) in the urine sample U, in particular determining Mxi-2 level in the urine sample U and optionally additionally the Vim3 level in the urine sample U;

determining whether the individual comprises a malignant kidney cancer or a benign oncocytoma; and

administering the individual with an amount of an antineoplastic agent sufficient for treating the malignant kidney cancer or the benign oncocytoma identified in the individual.

Optionally, in case of a benign oncocytoma, the individual may also be treated by removal of the kidney or preferably by resection of the benign oncocytoma, then preferably without administering an antineoplastic agent and without irradiation.

In another preferred embodiment, the method for treating an individual bearing (optionally suffering from) a neoplasm is a method for treating an individual bearing (optionally suffering from) a malignant kidney cancer comprising the following steps:
(i) providing a urine sample U obtained from the individual; and
(ii) determining Mxi-2 and/or Vim3 level(s) in the urine sample U, in particular determining Mxi-2 level in the urine sample U and optionally additionally the Vim3 level in the urine sample U;

determining whether the individual comprises a malignant kidney cancer; and administering the individual with an amount of an antineoplastic agent sufficient for treating the malignant kidney cancer in the individual.

In a highly preferred embodiment, the method for treating an individual bearing (optionally suffering from) a neoplasm comprises the following steps:
(i) providing a urine sample U obtained from the individual; and
(ii) determining Mxi-2 and/or Vim3 level(s) in the urine sample U, in particular determining Mxi-2 level in the urine sample U and optionally additionally the Vim3 level in the urine sample U;

determining whether the individual comprises a malignant kidney cancer or a benign oncocytoma; and

treating the individual in case of a malignant kidney cancer by administering the individual with an amount of an antineoplastic agent sufficient for treating the malignant kidney cancer and/or irradiation and/or excision (also: resection or enucleation) of the tumor and/or removal of the kidney;

or in case of a benign oncocytoma by removal of the kidney or preferably by resection of the benign oncocytoma (then preferably without administering an antineoplastic agent and without irradiation).

A person of ordinary skill in the art may recognize that a method of treatment of a renal neoplasm comprising a diagnostic step as described herein may spare the individual complete removal of the kidney or the side effects of the treatment with antineoplastic agents or irradiation.

It will be understood that all further specifications and preferred embodiments described in the context of the method for detecting malignant kidney cancer in an individual according to the invention as described herein also apply to such method for treating an individual.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain”, “include” and “encompass” are to be interpreted inclusively rather than exclusively. Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, some exemplified preferred methods and materials are described herein.

The following Examples as well as the accompanying Figures are intended to provide illustrative embodiments of the present invention described and claimed herein. These Examples and Figures are not intended to provide any limitation on the scope of the invented subject-matter.

### BRIEF DESCRIPTION OF THE FIGURES

FIG. 1 shows the comparison between renal cell carcinoma (RCC), oncocytoma (OC) and healthy control (Crt) urine samples. FIG. 1A shows the Mxi-2 level in RCC and OC urines in comparison to the controls (Ctr): Western blot analysis (above) and densitometric analysis of Western blots averaged from 10 independent experiments (below). The Mxi-2 level is particularly increased in RCC urine samples (***p<0.001). FIG. 1B shows the Vim3 level in RCC and OC urines in comparison to the controls (Ctr): Western blot analysis (above) and densitometric analysis of Western blots averaged from 10 independent experiments (below). The Vim3 level is particularly increased in OC urine samples (***p<0.001). All samples were neutralized to the housekeeping gene (β-actin).

FIG. 2 shows an exemplary setup of a dipstick usable for the method of the present invention. Such dipstick is usable for detecting and quantifying Mxi-2 or Vim3. FIG. 2A shows the dipstick before use. Herein, the urine sample U may be premixed with a labeled Mxi-2-specific (or Vim3-specific) antibodies (depicted as stars). S indicates the urine sample U to be added to the dipstick. (2) indicates a stripe comprising immobilized unlabeled Mxi-2-specific (or Vim3-specific, alternatively 38- or vimentin-specific) antibodies. (3) indicates a stripe of immobilized unlabeled antibodies specifically binding the labeled Mxi-2-specific (or Vim3-specific) antibodies (depicted as stars). (4) indicates the flow direction of the moisten urine sample U. FIG. 2b shows the results when the respective Mxi-2-containing (or Vim3-containing) sample (S+) premixed with a labeled Vim3-specific antibodies (depicted as stars) is added to the dipstick. Then, the labeled Mxi-2-specific (or Vim3-specific) antibodies (depicted as stars) and the Mxi-2 (or Vim3) in the urine sample U form a Mxi-2:Mxi-2-specific antibody (or Vim3:Vim3-specific antibody) conjugate. After adding this urine sample U to the dipstick, it flows through the dipstick (4). The antibody conjugate is then binding to the unlabeled Mxi-2-specific (or Vim3-specific) antibodies of stripe (2). FIG. 2c shows the results when a sample lacking Mxi-2 (or Vim3) (S-) premixed with a labeled Mxi-2-specific (or Vim3-specific) antibodies (depicted as stars) is added to the dipstick. Then, the labeled Mxi-2-specific (or Vim3-specific) antibodies (depicted as stars) are not binding to its target. Thus, upon flowing through the dipstick (4), the labeled Mxi-2-specific (or Vim3-specific) antibodies are not bound until the stripe (3). Thus, the ratio between signal intensity of the label in stripe (2) and (3) indicates the presence of Mxi-2 (or Vim3). A higher (2):(3) ratio indicates higher level of Mxi-2 (or Vim3), whereas a lower (2):(3) ratio indicates lower level.

FIG. 3 shows another setup of a dipstick usable for the method of the present invention as an alternative to the one shown in FIG. 2. FIG. 3A shows the dipstick before use. Herein, (1) indicates a stripe comprising labeled Mxi-2-specific (or Vim3-specific) (depicted as stars), which are not immobilized and freely movable when the urine sample U passes through this stripe. (2) indicates a stripe comprising immobilized unlabeled Mxi-2-specific (or Vim3-specific) antibodies. (3) indicates a stripe of immobilized unlabeled antibodies specifically binding the labeled Mxi-2-specific (or Vim3-specific) antibodies (depicted as stars). S indicates the urine sample U to be added to the dipstick. (4) indicates the flow direction of the moisten urine sample U. FIG. 3b shows the results when a Mxi-2-containing (or Vim3-containing) sample (S+) is added to the dipstick. Then, upon flowing through the dipstick (4), the labeled Mxi-2-specific (or Vim3-specific) antibodies are binding to Mxi-2 (or Vim3) in the urine sample U and form a Mxi-2:Mxi-2-specific antibody (or Vim3:Vim3-specific antibody) conjugate. This conjugate is then binding to the unlabeled Mxi-2-specific (or Vim3-specific) antibodies of stripe (2). FIG. 3c shows the results when a sample lacking Mxi-2 (or Vim3) (S-) is added to the dipstick. Then, upon flowing through the dipstick (4), the labeled Mxi-2-specific (or Vim3-specific) antibodies are not bound until the stripe (3). Thus, the ratio between signal intensity of the label in stripe (2) and (3) indicates the presence of Mxi-2 (or Vim3). A higher (2):(3) ratio indicates higher level of Mxi-2 (or Vim3), whereas a lower (2):(3) ratio indicates lower level.

FIG. 4 shows an example of a dipstick usable for the method of the present invention for concomitantly detecting Mxi-2 and Vim3. This reflects a dipstick as shown in FIG. 3, wherein each of stripes (1), (2) and (3) are each present twice, each for detecting Mxi-2 (indicated by stars) and for detecting Vim3 (indicated by x). The definitions are corresponding to those indicated in the context of FIGS. 2 and 3 above. It will be understood that stripe (3) can alternatively also be represented by a single stripe of immobilized unlabeled antibodies concomitantly binding the labeled Mxi-2-specific and Vim3-specific antibodies (depicted as stars and x) when these have the same or a comparable Fc fragment. It will be understood that the stripes (1) and (1′) may alternatively also form a single stripe comprising a mixture of Mxi-2-specific antibodies and Vim3-specific antibodies.

FIG. 5 shows the comparison of Mxi-2 levels in different urine samples by means of an ELISA assay. Urine samples from healthy individuals (control, average of samples from ten individuals) were compared to urine samples from individuals having chromophobe RCC (chromo RCC, average of samples from eight individuals), eosinophilic RCC (eosino RCC, average of samples from four individuals), papillary RCC (pap RCC, average of samples from eight individuals), oncocytoma (average of samples from eight individuals), and RCC (average of samples from ten individuals). The Mxi-2 level is increased in RCC urine samples in comparison to samples from healthy controls (**p<0.001) and samples from individuals having an oncocytoma (**p<0.001), but is not significantly altered in a sample from an individual having an oncocytoma. The dashed line indicates the average of the control sample.

FIG. 6 shows the comparison of MAPK p38 levels in different urine samples by means of an ELISA assay. Urine samples from healthy individuals (control, average of samples from ten individuals) were compared to urine samples from individuals having chromophobe RCC (chromo RCC, average of samples from eight individuals), eosinophilic RCC (eosino RCC, average of samples from four individuals), papillary RCC (pap RCC, average of samples from eight individuals), oncocytoma (average of samples from eight individuals), and RCC (average of samples from ten individuals). The MAPK p38 level is increased in RCC urine samples in comparison to samples from healthy controls (*p<0.001), but is not significantly altered in a sample from an individual having an oncocytoma. Increase is comparably lower in papillary RCC. The dashed line indicates the average of the control sample.

FIG. 7 shows the comparison of Vim3 levels in different urine samples by means of an ELISA assay. Urine samples from healthy individuals (control, average of samples from ten individuals) were compared to urine samples from individuals having chromophobe RCC (chromo RCC, average of samples from eight individuals), eosinophilic RCC (eosino RCC, average of samples from four individuals), papillary RCC (pap RCC, average of samples from eight individuals), oncocytoma (average of samples from eight individuals), and RCC (average of samples from ten individuals). The Vim3 level is increased in oncocytoma samples in comparison to samples from healthy controls and various RCC samples (*p, **p and ***p<0.001), but is not significantly altered in a sample from an individual having an RCC of any type. The dashed line indicates the average of the control sample.

FIG. 8 shows the comparison of Atg7 levels in different urine samples by means of an ELISA assay. Urine samples from healthy individuals (control, average of samples from ten individuals) were compared to urine samples from individuals having chromophobe RCC (chromo RCC, average of samples from eight individuals), eosinophilic RCC (eosino RCC, average of samples from four individuals), papillary RCC (pap RCC, average of samples from eight individuals), oncocytoma (average of samples from eight individuals), and RCC (average of samples from ten individuals). The Atg7 is increased in RCC urine samples in comparison to samples from healthy controls (*p and **p<0.001), but is not significantly altered in a sample from an individual having an oncocytoma. Increase is comparably low in papillary RCC. The dashed line indicates the average of the control sample.

### EXAMPLES

### Example I—Western Blot Analysis

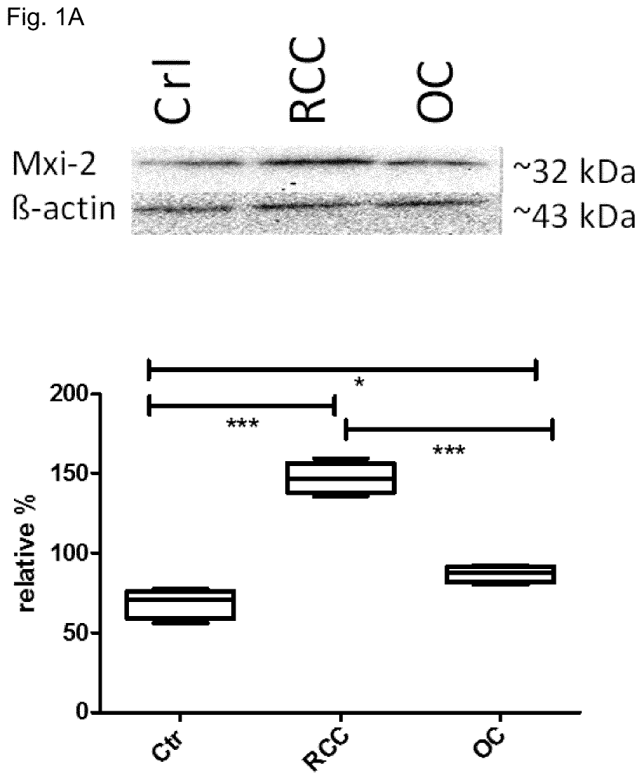

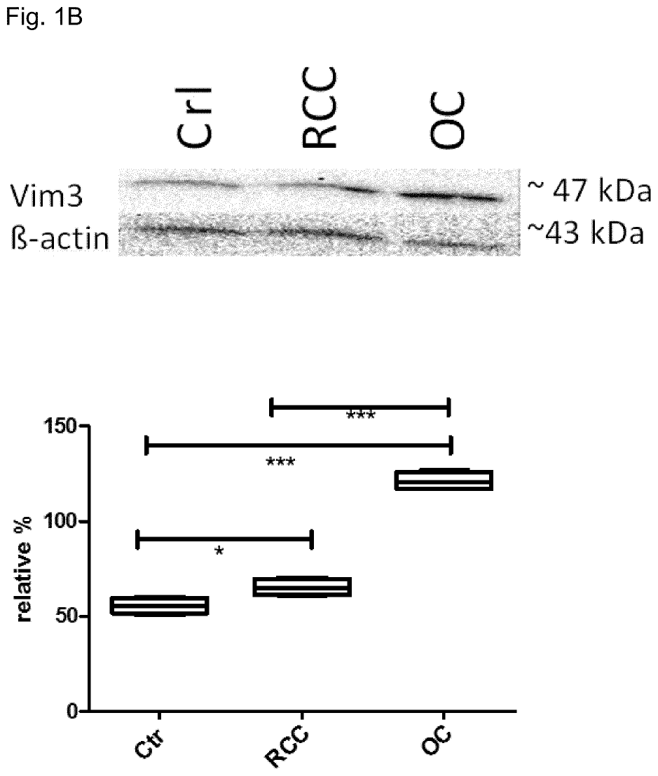

Western blot analysis of urine samples from patients with either RCC or oncocytoma (OC) compared with “healthy” control urine samples (Crt). All samples were neutralized to beta-actin (β-actin) used as housekeeping gene. For densitometry (FIGS. 1A and 1B), each 10 patient samples were measured. The background of the blot was always measured separately and calculated against the densitometry of the protein lane. Afterwards, the results from the housekeeping gene were determined as 100% and either the Mxi-2 or Vim3 signal was calculated in regard to the 100% of the housekeeping. Calculation was performed by 1st way ANOVA.

Western blot was performed as followed: Proteins were separated according to their molecular weight. This was done by SDS polyacrylamide gel electrophoresis (SDS-PAGE) in a discontinuous gel system to enhance the sharpness of the bands within the gel. The discontinuous gel system is composed of a stacking and a separating gel which differs in salt concentration, pH and acrylamide concentration. A 10% separating gel was used and performed as followed: the separating gel contains 0.4 M Tris-HCl pH 8.8, 0.1% SDS, 10-12% acrylamide/bisacrylamide (29:1), 0.5% ammonium persulfate and 0.06% TEMED. The stacking gel contains 0.125 M Tris pH 6.8, 0.1% SDS, 3% acrylamide/bisacrylamide (29:1), 0.5% ammonium persulfate and 0.12% TEMED.

To load the gel, 500 to 1000 μl urine were centrifuged at full speed and resuspended with PBS and “5× Western loading dye” and heated for 5 min at 99° C. The gel run was performed in “Laemmlis running” buffer. The running time was between 1.5 h to 2 h (15 mA until dye front reached separating gel, then 30 mA) depending on the protein size. After separation in the SDS-PAGE the proteins were transferred onto PVDF membrane using a semidry blotting system in Towbin buffer. 9 layers of 3 MM Whatman paper were placed in the semidry chamber with the gel on top. The gel was covered by an activated PDVF membrane. Finally 9 further layers of Whatman paper were placed on top. The 3 MM Whatman paper was previously moistened in “Towbin buffer”. The PDVF membrane was activated according to the manufacturing protocol. The transfer proceeded at 1.2 mA/cm2 at 4° C. for exactly 70 min.

After the transfer the membrane was blocked by putting it in blocking solution (5% milk in TBST) for 1 hour, shaking at room temperature, to avoid unspecific binding of the primary antibody. Following that, the membrane was incubated with the first antibody in blocking solution (5% milk in TBST) over night at 4° C., shaking. After this time and after a 30 min washing step in TBST stock solution, the membrane was incubated with the secondary antibody in blocking solution (5% milk in TBST) for 1 hour, shaking at room temperature and then washed again for 30 min in TBST stock solution. Finally the membrane was incubated 1.5 min with ECL reagent and developed in a chemiluminescence reader (ChemiDoc, Biorad).

The antibodies usable in the context of the method of the present invention are exemplarily specifically binding to the following epitopes:

epitope present at a region of Mxi-2:

GKLTIYPHLMDIELVMI (SEQ ID NO: 4)

epitope present at the C-terminal region of Vim3:

NLRGKHFISL (SEQ ID NO: 5)

It has been found surprisingly that individuals suffering from renal cell carcinoma (RCC) show increased levels of Mxi-2, whereas individuals bearing oncocytoma show increased levels of Vim3.

### Example II—Analysis Via Dipstick

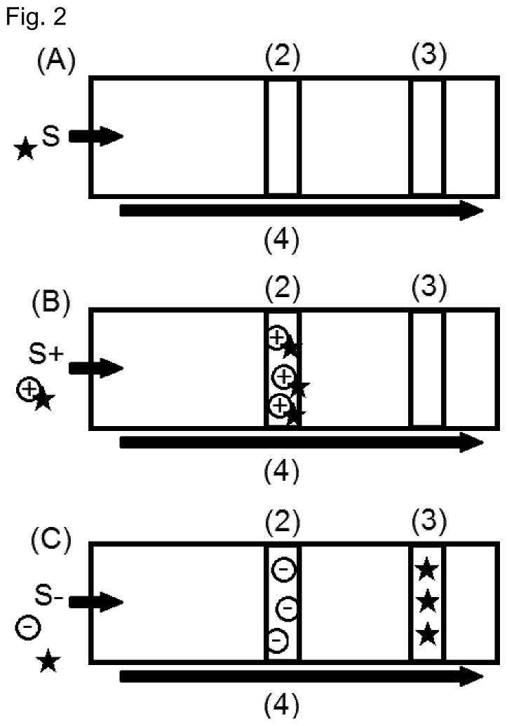

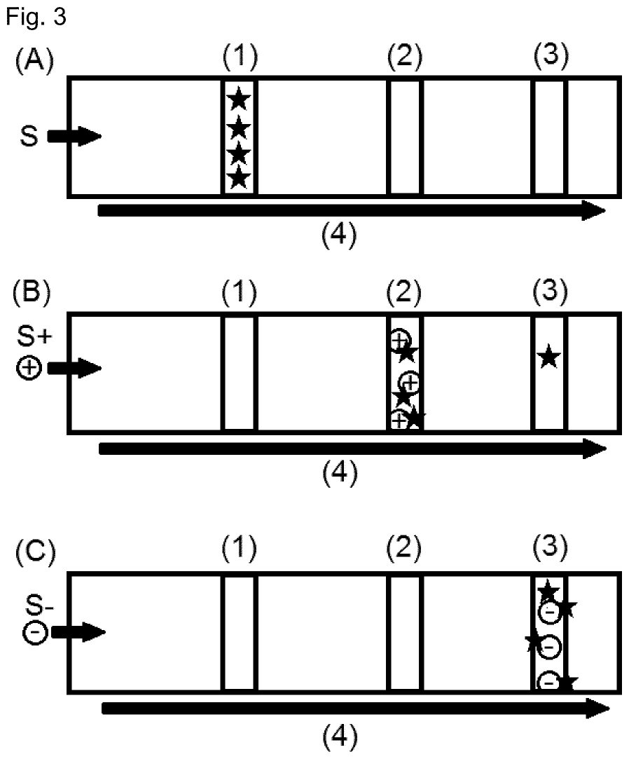

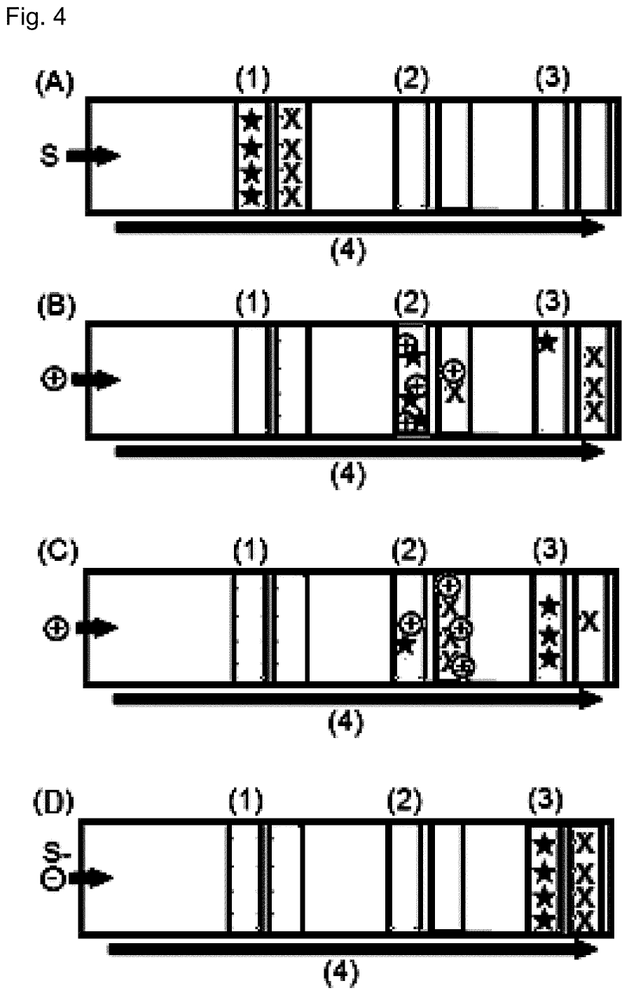

For dipstick analysis the same procedure can be used as for the sample analysis, at least when a single dipstick test is performed (Mxi-2 or Vim3). This is further exemplified in FIGS. 2 and 3. Also a combinational (i.e., combined) dipstick for the concomitant/parallel analysis of Mxi-2 and Vim3 in one urine sample U is usable. This is further exemplified in FIG. 4. This analysis indicates the level of Mxi-2 and Vim3, respectively, and optionally the ratio of Mxi-2:Vim3. The levels found in a urine sample U of interest are comparable with predetermined threshold level(s) and/or with those levels determined in one or more control samples.

### Example III—Analysis Via Enzyme-Linked Immunosorbent Assay (ELISA)

Furthermore, also an ELISA is usable in the context of the present invention. For this purpose, an ELISA plate was labelled with an antibody which detects both variants, the full length and the truncated. ELISA plates (Corning Costar®96-Well EIA/RIA Stripwell™ Plates) were washed before start 2× with. 100 μl of the urine sample was added to the plate and incubated for 1 hour. After incubation plates were washed 2× with PBS and incubated for 1 h at room temperature with the primary antibody (1:1000) □ Atg7 (Santa Cruz, H-300; Vim3 monoclonal, clon 51, Davids Lab; Mxi-2, Nanotools, clon 2F2; MAPK p38, Santa Cruz, C-20). The samples were washed again with PBS 2× (alternatively 3×) and incubated with the corresponding, labelled secondary antibody. The signal detection depends on the used secondary antibody used. More in detail, after incubation plates were washed 2× with PBS and incubated for 1 h at room temperature with the primary antibody (1:1000) Atg7 (Santa Cruz, H-300; Vim3 monoclonal, clone 51, Davids Lab; Mxi-2, Nanotools, clone 2F2; MAPK p38, Santa Cruz, C-20). After incubation plates were washed again with PBS 2× and incubated for 1 h at room temperature with secondary goat-anti mouse antibody (1:50000) (Columbia Biosciences, HRP112). After final incubation step ELISAs were washed 3× with PBS and TMB solution was add for 10 min. afterwards the reaction was stopped with ELISA stopping solution and plates were analysed at 450 nm. The levels found in a urine sample U of interest were comparable with predetermined threshold level(s) and/or with those levels determined in one or more control samples.

Urine samples from healthy individuals (control, samples from ten individuals) were compared to urine samples from individuals having chromophobe RCC (samples from eight individuals), eosinophilic RCC (samples from four individuals), papillary RCC (samples from eight individuals), oncocytoma (samples from eight individuals), and RCC (samples from ten individuals).

ELISA assays were performed to determine the levels of Mxi-2, MAPK p38, Atg7 and Vim3 in the urine samples.

The results obtained by ELISA analysis confirmed the results performed by Western Blot analysis above. It was found that the Mxi-2 level, the MAPK p38 level and the Atg7 level in urine samples obtained from individuals suffering from RCC is statistically significantly increased in comparison to the levels found in urine samples from healthy control individuals and individuals having an oncocytoma.

Remarkably, the Mxi-2 level, the MAPK p38 level and the Atg7 level determined in urine samples obtained from individuals having chromophobe RCC or eosinophilic RCC were similar to those determined in urine samples obtained from individuals having RCC. The MAPK p38 level and the Atg7 level determined in urine samples obtained from individuals having papillary RCC was somewhat lower. Papillary RCC is typically considered as being averagely somewhat less malignant and having a better prognosis than chromophobe RCC, eosinophilic RCC or common RCC. This indicates that the presently claimed assay indicates the malignancy of a malignant renal carcinoma.

It was found that the Vim3 level in urine samples obtained from individuals having an oncocytoma is statistically significantly increased in comparison to the levels found in urine samples from healthy control individuals and individuals having an RCC.

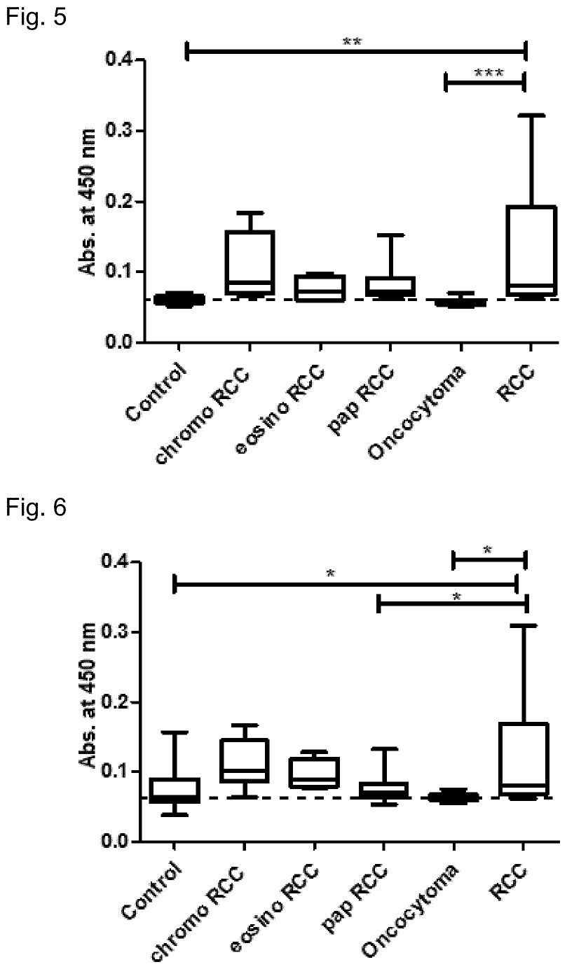

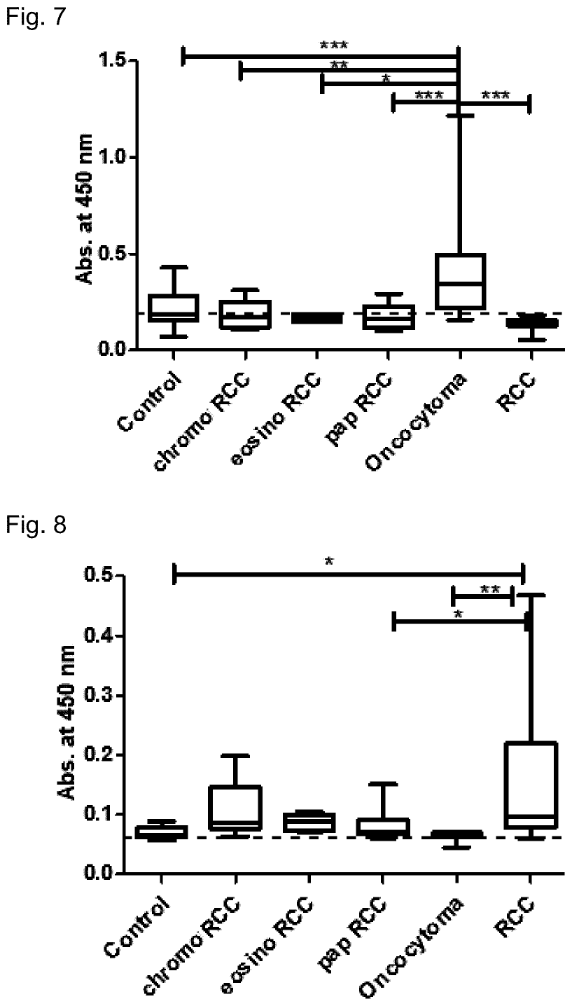

The comparison between the levels of Mxi-2 in urine (see FIG. 5) and MAPK p38 (see FIG. 6) further shows that the Mxi-2 antibody appears to be even more specific. The statistic deviations between control samples are smaller for the Mxi-2 antibody.

### Example IV—Analysis Via Polymerase Chain Reaction (PCR)

Furthermore, also PCR is usable in the context of the present invention. For PCR analysis the following primers are exemplarily usable:

Mxi-2:
Forward:
(SEQ ID NO: 6)
5′-GACTCAGATGCCGAAGAT-3′
Reverse:
(SEQ ID NO: 7)
5′-TCAACTAATGGTACTTTATTTGG-3′
Vim3:
Forward:
(SEQ ID NO: 8)
5′-GAGAACTTTGCCGTTGAAGC-3′
Reverse:
(SEQ ID NO: 9)
5′-GAAATAAAATGCTTACCCCTCAG-3′

The levels found in a urine sample U of interest are comparable with predetermined threshold level(s) and/or with those levels determined in one or more control samples.

1. A method for detecting malignant kidney cancer in an individual, said method conducted in vitro, comprising determining an Mxi-2 level in a urine sample U from the individual.

2. The method of claim 1, comprising the following steps:
(i) providing a urine sample obtained from the individual; and
(ii) determining the Mxi-2 level in the urine sample.

3. The method of claim 1, wherein the malignant kidney cancer is renal cell carcinoma.

4. The method of claim 1, wherein the step of determining the Mxi-2 level is determining a level of Mxi-2 polypeptide.

5. The method of claim 1, wherein the step of determining the Mxi-2 level is determining a level of Mxi-2 messenger RNA.

6. The method of claim 1, wherein an increased Mxi-2 level indicates a presence of malignant kidney cancer in the individual.

7. The method of claim 2, wherein the method further comprises the step of:
(iii) comparing the Mxi-2 level determined in step (ii) with
(a) a predetermined reference value (R1) indicating a borderline between a sample indicating a presence of malignant kidney cancer and a sample indicating an absence of malignant kidney cancer;
(b) an Mxi-2 level determined in a control sample obtained from a control individual of the same species known to be free of malignant cancer cells; or
(c) a combination of (a) and (b),
wherein an Mxi-2 level determined in the urine sample that is higher than R1, at least 20% higher than the Mxi-2 level of the control sample, or both indicates the presence of malignant kidney cancer in the individual,
wherein the Mxi-2 level in each case is related to a total polypeptide content comprised in the respective sample.

8. The method of claim 1, wherein the method further comprises the step of
determining a Vimentin variant 3 (Vim3) level in the urine sample.

9. The method of claim 8, wherein the method further comprises the step of
(v) comparing the Vim3 level determined in step (iv) with
(a) a predetermined reference value (R2) indicating a borderline between a sample indicating a presence of malignant kidney cancer and a sample indicating an absence of malignant kidney cancer;
(b) Vim3 level determined in the control sample C; or
(c) a combination of (a) and (b),
herein an Vim3 level determined in the urine sample that is lower than R2, that is at least 50% lower than the Vim3 level of the control sample, or that is lower than R2 and at least 50% lower than the Vim3 level of the control sample indicates the presence of malignant kidney cancer in the individual,
wherein the Vim3 level in each case is related to a total polypeptide content comprised in the respective sample.

10. The method of 8, wherein an increase of a ratio of Mxi-2:Vim3 levels in comparison to a predetermined reference value R3 indicating a borderline between a sample indicating a presence of malignant kidney cancer and a sample indicating an absence of malignant kidney cancer indicates the presence of malignant kidney cancer in the individual.

11. The method of claim 2, wherein step (ii) and if present step (iv) comprises at least one of
a) determining a respective polypeptide level of Mxi-2, Vim3, or both by means of conducting at least one step selected from the group consisting of enzyme-linked immunosorbent assay (ELISA), immuno-electrophoresis, immuno-blotting, Western blot, SDS-PAGE, capillary electrophoresis (CE), spectrophotometry or enzyme assay for example, dipsticks (lateral flow), and combinations of two or more thereof;
b) determining a respective messenger RNA level of Mxi-2, Vim3, or both by means of conducting at least one step selected from the group consisting of polymerase chain reaction (PCR), real time PCR (RT-PCR), by in situ hybridization, gel electrophoresis, Northern Blot, Southern Blot, and combinations of two or more thereof; or
c) determining the respective polypeptide level and the respective messenger RNA level of Mxi-2, Vim3, or both according to a) and b).

12. The method of claim 2, wherein step (ii) and if present step (iv) comprises staining of the respective polypeptide Mxi-2, Vim3, or both.

13. A kit for use in a method according to claim 8, comprising:
(A) means for determining the Mxi-2 level in an urine sample;
(B) means for determining the Vim3 level in an urine sample; and
(C) instructions for carrying out the method according to claim 8.

14. A dipstick usable for the method of claim 1 comprising, placed in the direction of flow of the urine sample, on a carrier that is suitable for soaking the urine sample, the following:
(0) an edge or segment suitable for receiving the urine sample;
(1) optionally a stripe (1) comprising labeled Mxi-2-specific antibodies or antibody fragments which are not immobilized and freely movable when the urine sample passes through this stripe (1);
(2) a stripe (2) comprising immobilized unlabeled MAPK p38 or Mxi-2-specific antibodies or antibody fragments; and
(3) optionally a stripe (3) of immobilized unlabeled antibodies or antibody fragments specifically binding the labeled Mxi-2-specific antibodies or antibody fragments of stripe (1).

15. A dipstick usable for the method of claim 8 comprising, placed in the direction of flow of the urine sample, on a carrier that is suitable for soaking the urine sample, the following:
(0) an edge or segment suitable for soaking the urine sample U;
(1) optionally a stripe (1) comprising labeled Mxi-2-specific antibodies or antibody fragments which are not immobilized and freely movable when the urine sample passes through this stripe (1);
(1′) optionally a stripe (1′) comprising labeled Vim3-specific antibodies or antibody fragments which are not immobilized and freely movable when the urine sample U passes through the one or more stripe(s) (1);
(2) a stripe (2) comprising immobilized unlabeled MAPK p38-specific or Mxi-2-specific antibodies or antibody fragments;
(2′) a stripe (2′) comprising immobilized unlabeled vimentin-specific or Vim3-specific; antibodies or antibody fragments;
(3) optionally a stripe (3) of immobilized unlabeled antibodies or antibody fragments specifically binding the labeled Mxi-2-specific antibodies or antibody fragments of stripe (1); and
(3′) optionally a stripe (3′) of immobilized unlabeled antibodies or antibody fragments specifically binding the labeled Vim3-specific antibodies or antibody fragments of stripe (1).

16. An antineoplastic agent for use in a method for treating an individual bearing malignant kidney cancer, wherein the malignant kidney cancer has previously been detected in the individual by means of a method of claim 1.

17. An antibody or fragment or variant thereof specific for Mxi-2 polypeptide.

18. A cell capable of producing the antibody or fragment or variant thereof according to claim 17.

19. A method for detecting an oncocytoma in an individual, said method conducted in vitro, comprising determining a Vim3 level in a urine sample from the individual.

20. The method of claim 19, comprising the following steps:
(i) providing a urine sample obtained from the individual; and
(ii) determining Vim3 level in the urine sample.

21. The method of claim 20, wherein the method further comprises the step of
(iii) comparing the Vim3 level determined in step (ii) with
(a) a predetermined reference value R1 indicating a borderline between a sample indicating a presence of a oncocytoma and a sample indicating an absence of malignant kidney cancer;
(b) Vim3 level determined in a control sample C-obtained from a control individual of the same species free of an oncocytoma; or
(c) a combination of (a) and (b),
wherein an Vim3 level determined in the urine sample that is higher than R1, at least 20% higher than the Vim3 level of, or both indicates the presence of an oncocytoma in the individual,
wherein the Vim3 level in each case is related to the total polypeptide content comprised in the respective sample.

22. The method of claim 1, wherein the method further comprises the step of
(iv) determining a Vimentin variant 3 (Vim3) level in the urine sample, wherein
determining the Vim3 level comprises determining the level of Vim3 polypeptide, the level of Vim3 messenger RNA, or both.

23. The method of claim 1, wherein the method further comprises the step of
(iv) determining a Vimentin variant 3 (Vim3) level in the urine sample,
wherein a decreased Vim3 level or the absence of Vim3 indicates the presence of malignant kidney cancer in the individual.

24. The method of 10, wherein the individual is known to comprise either
(a) malignant renal cell carcinoma, or
(b) benign oncocytoma,
and the method is conducted to differentiate between (a) and (b),
wherein (a) is characterized by an increase of a ratio Mxi-2:Vim3 levels.

25. The method of claim 2, wherein step (ii) and if present step (iv) comprises staining of the respective polypeptide Mxi-2, Vim3, or both by:
(iia) direct immunodetection comprising providing at least one labeled antibody or antibody fragment (AB1-L) specific for the respective polypeptide, and
enabling the binding of said AB1-L to the respective polypeptide; or
(iib) indirect immunodetection comprising providing at least one unlabeled antibody or antibody fragment (AB1-ul) specific for the respective polypeptide and at least one labeled antibody or antibody fragment (AB2-L) specifically binding to AB1-ul, enabling the binding of AB1-ul to the respective polypeptide, and
enabling the binding of AB2-L to AB1-ul.

26. The antibody or fragment or variant thereof of claim 17, wherein said antibody or fragment or variant thereof binds to the Mxi-2 polypeptide with a dissociation constant of not more than 20 nM and to full length MAPK p38 with a dissociation constant of more than 20 nM.


SEQUENCE LISTING (12 sequences)

SEQ ID NO: 1 - protein, 431 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRGKHFISL

SEQ ID NO: 2 - protein, 8 aa, Homo sapiens
RGKHFISL

SEQ ID NO: 3 - protein, 297 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLGKLTIYPHLMDIELVMI

SEQ ID NO: 4 - protein, 17 aa, Homo sapiens
GKLTIYPHLMDIELVMI

SEQ ID NO: 5 - protein, 10 aa, Homo sapiens
NLRGKHFISL

SEQ ID NO: 6 - DNA, 18 bp, Artificial Sequence
  Mxi-2 primer forward
gactcagatgccgaagat

SEQ ID NO: 7 - DNA, 23 bp, Artificial Sequence
  Mxi-2 primer reverse
tcaactaatggtactttatttgg

SEQ ID NO: 8 - DNA, 20 bp, Artificial Sequence
  Vim3 primer forward
gagaactttgccgttgaagc

SEQ ID NO: 9 - DNA, 23 bp, Artificial Sequence
  Vim3 primer reverse
gaaataaaatgcttacccctcag

SEQ ID NO: 10 - DNA, 1431 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttga

SEQ ID NO: 11 - protein, 703 aa, Homo sapiens
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 12 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES